(19) United States
(12) Patent Application Publication
(10) Pub. No.: US 2023/0313287 A1
(43) Pub. Date: Oct. 5, 2023

(54) SYSTEMS AND METHODS FOR NUCLEIC ACID SEQUENCING

(71) Applicant: Ultima Genomics, Inc., Newark, CA (US)

(72) Inventors: Gilad ALMOGY, Newark, CA (US); Linda LEE, Palo Alto, CA (US)

(21) Appl. No.: 17/991,429

(22) Filed: Nov. 21, 2022

Related U.S. Application Data
(63) Continuation of application No. 16/358,185, filed on Mar. 19, 2019, now abandoned.
Application No. 16/358,185 is a continuation of application No. PCT/US2017/053948, filed on Sep. 28, 2017.
(60) Provisional application No. 62/401,670, filed on Sep. 29, 2016.
(60) Provisional application No. 62/466,007, filed on Mar. 2, 2017.

(51) Int. Cl. C12Q 1/6869

(52) U.S. Cl. CPC C12Q 1/6869

(57) ABSTRACT

The present disclosure provides methods and systems for determining a nucleic acid sequence of a target nucleic acid molecule. A method for sequencing a target nucleic acid molecule comprises subjecting a plurality of nucleic acid molecules exhibiting sequence homology to the target nucleic acid molecule to at most 4000 cycles a nucleic acid extension reaction while measuring detectable signals from the plurality of nucleic acid molecules. The detectable signals may correspond to individual nucleotides or nucleotide analogs incorporated into the plurality of nucleic acid molecules during the nucleic acid extension reaction. The detectable signals may be used to generate a sequence of the target nucleic acid molecule at a length of at least about 500 bases and an accuracy of at least about 97%.

### CROSS-REFERENCE TO RELATED APPLICATIONS

This application is a continuation of U.S. patent application Ser. No. 16/358,185, filed Mar. 19, 2019, which is a continuation of International Patent Application No. PCT/US2017/053948, filed Sep. 28, 2017, which claims the benefit of U.S. Provisional Patent Application No. 62/401,670, filed Sep. 29, 2016, and U.S. Provisional Patent Application No. 62/466,007, filed Mar. 2, 2017, which applications are herein incorporated by reference in its entirety for all purposes.

### BACKGROUND

The detection, quantification and sequencing of nucleic acid molecules (e.g., polynucleotides) may be important for molecular biology and medical applications, such as diagnostics. Genetic testing is particularly useful for a number of diagnostic methods. For example, disorders that are caused by rare genetic alterations (e.g., sequence variants) or changes in epigenetic markers, such as cancer and partial or complete aneuploidy, may be detected or more accurately characterized with deoxyribonucleic acid (DNA) or ribonucleic acid (RNA) sequence information.

The goal to elucidate the entire human genome has created interest in technologies for rapid nucleic acid (e.g., DNA) sequencing, both for small and large scale applications. Important parameters are sequencing speed, sequencing accuracy, length of sequence that can be read during a single sequencing run, and amount of nucleic acid template required to generate sequencing information. Large scale genome projects are currently too expensive to realistically be carried out for a large number of subjects (e.g., patients). Furthermore, as knowledge of the genetic basis for human diseases increases, there will be an ever-increasing need for accurate, high-throughput DNA sequencing that is affordable for clinical applications. Practical methods for determining the base pair sequences of single molecules of nucleic acids, preferably with high speed, high accuracy and long read lengths, may provide measurement capability.

Nucleic acid sequencing is a process that can be used to provide sequence information for a nucleic acid sample. Such sequence information may be helpful in diagnosing and/or treating a subject with a condition. For example, the nucleic acid sequence of a subject may be used to identify, diagnose and potentially develop treatments for genetic diseases. As another example, research into pathogens may lead to treatment for contagious diseases. Unfortunately, though, existing sequencing technology of the status quo is expensive and may not provide sequence information within a time period and/or at an accuracy that may be sufficient to diagnose and/or treat a subject with a condition.

### SUMMARY

Recognized herein is a need for systems and methods for high throughput nucleic acid sequencing with high accuracy. The present disclosure provides systems and methods for nucleic acid sequencing. Such systems and methods may enable sequences of relatively long lengths to be obtained.

An aspect of the disclosure provides a method for determining a nucleic acid sequence of a target nucleic acid molecule. The method comprises: (a) providing a plurality nucleic acid molecules immobilized to a support, where each of the plurality of nucleic acid molecules exhibits sequence homology to the target nucleic acid molecule, and where the support is operatively coupled to a detector; (b) directing a plurality of nucleotides or nucleotide analogs to the support, which plurality of nucleotides or nucleotide analogs comprises at least a first subset of nucleotides or nucleotide analogs and a second subset of nucleotides or nucleotide analogs, where (i) each of the first subset of nucleotides or nucleotide analogs comprises a detectable moiety and a terminating subunit, and (ii) none of the second subset of nucleotides or nucleotide analogs comprises the detectable moiety and the terminating subunit; (c) incorporating the plurality of nucleotides or nucleotide analogs comprising the first subset of nucleotides or nucleotide analogs and the second subset of nucleotides or nucleotide analogs into the plurality nucleic acid molecules, where during incorporation, a given nucleotide or nucleotide analog from the first subset of nucleotides or nucleotides analogs is incorporated into a given nucleic acid molecule from the plurality of nucleic acid molecules, which given nucleotide or nucleotide analog comprises the detectable moiety and the terminating subunit; and (d) using the detector to detect the detectable moiety from the given nucleotide or nucleotide analog, thereby determining the nucleic acid sequence of the target nucleic acid molecule.

In some embodiments, a ratio of the first subset of nucleotides or nucleotide analogs to the second subset of nucleotides or nucleotide analogs is less than 50%, less than 10%, less than 1%, less than 0.001% or less than 0.0001%. In some embodiments, the ratio is 1 base/x, where ‘x’ is a length of a sequence read corresponding to the nucleic acid sequence.

In some embodiments, the target nucleic acid molecule is a deoxyribonucleic acid molecule. In some embodiments, the method further comprises subjecting the target nucleic acid molecule to nucleic acid amplification (e.g., polymerase chain reaction, emulsion-based amplification, bridge amplification) to generate the plurality nucleic acid molecules. In some embodiments, the target nucleic acid molecule is a ribonucleic acid molecule. In such embodiments, the method can further comprise subjecting the target nucleic acid molecule to reverse transcription to generate the plurality nucleic acid molecules.

In some embodiments, the support is a solid support, a flow cell or an open substrate. The support can also be a biological support, non-biological support, organic support, inorganic support, or any combination thereof. In some embodiments, the detectable moiety is optically detectable, such as a fluorophore. In some embodiments, the detectable moiety is an acceptor or a donor. In some embodiments, the detectable moiety is detected via Förster resonance energy transfer (FRET).

In some embodiments, the plurality of nucleotides or nucleotide analogs include deoxynucleotides or dideoxynucleotides. In some embodiments, the plurality of nucleotides or nucleotide analogs are selected from among the group of deoxyadenosine triphosphate (dATP), 2′,3′-ideoxyadenosine-5′-triphosphate (ddATP), deoxyguanosine triphosphate (dGTP), 2′,3′-dideoxyguanosine-5′-triphosphate (ddGTP), deoxycytidine triphosphate (dCTP), 2′,3′-dideoxycytidine-5′-triphosphate (ddCTP), deoxythymidine triphosphate (dTTP), 2′,3′-dideoxythymidine-5′-triphosphate (ddTTP), deoxyuridine triphosphate (dUTP), 2′,3′-dideoxyuridine-5′-triphosphate (ddUTP), or a variant thereof.

In some embodiments, the first subset of nucleotides or nucleotide analogs comprises a nucleotide or nucleotide analog with an unblocked 3′ hydroxyl. In some embodiments, the nucleotide or nucleotide analog with the unblocked 3′ hydroxyl is a 2-nitrobenzyl-modified thymidine analog.

In some embodiments, the method further comprises cleaving, bleaching, quenching or disabling the detectable moiety. The detectable moiety can be cleaved, bleached, quenched or disabled subsequent to detecting the detectable moiety from the given nucleotide or nucleotide analog. For example, cleaving can be chemical, enzymatic, or light induced. In some embodiments, the plurality of nucleotides or nucleotide analogs includes bases of the same type.

In some embodiments, the method further comprises repeating (b)-(d). The ratio can be modified every repetition or after a fixed number of repetitions. In some embodiments, the ratio is a function of the detector detecting detectable moieties, is pre-designated and/or is algorithmically calculated along the read. In some embodiments, the plurality of nucleotides or nucleotide analogs include bases of a first type, and (b)-(d) are repeated with an additional plurality of nucleotides or nucleotide analogs including bases of a second type different than the first type. The additional plurality of nucleotides or nucleotide analogs can include a third subset of nucleotides or nucleotide analogs, each of which third subset of nucleotides or nucleotide analogs having an additional detectable moiety different than the detectable moiety.

In some embodiments, when (b)-(d) are repeated, (d) comprises determining a first signal indicative of incorporation of the given nucleotide or nucleotide analog, comparing the first signal indicative of incorporation of the given nucleotide or nucleotide analog to a second signal indicative of incorporation of a previous nucleotide or nucleotide analog incorporated before the given nucleotide or nucleotide analog, and comparing a difference in the first signal and second signal to one or more predetermined signals indicative of incorporation for the given nucleotide or nucleotide analog comprising the detectable moiety and the terminating subunit to determine the nucleic acid sequence of the target nucleic acid molecule.

In some embodiments, (b)-(d) are repeated without cleaving the terminating subunit. In some embodiments, the detectable moiety is an optically detectable moiety and (d) comprises spectrally shifting an excitation wavelength of the detectable moiety. In some embodiments, the plurality of nucleotides or nucleotide analogs is incorporated using a nucleic acid polymerizing enzyme. The nucleic acid polymerizing enzyme can be a deoxyribonucleic acid polymerase, such as phi-29 or a variant thereof. In some embodiments, the detectable moiety is detected while incorporating the given nucleotide or nucleotide analog into the given nucleic acid molecule. In some embodiments, the detectable moiety is detected subsequent to incorporating the given nucleotide or nucleotide analog into the given nucleic acid molecule. In some embodiments, the detectable moiety is detected during or subsequent to incorporating the given nucleotide or nucleotide analog into the given nucleic acid molecule and washing unincorporated nucleotides or nucleotide analogs among the plurality of nucleotides or nucleotide analogs.

In some embodiments, the support is in optical communication with the detector and/or may have a plurality of independently addressable locations. The plurality nucleic acid molecules can be immobilized to the support at a given independently addressable location of the plurality of independently addressable locations. In some embodiments, the support is optically coupled to the detector. In some embodiments, the support is a bead and the detector is configured to maintain substantially the same read rate independent of a size of the bead. In some embodiments, each of the plurality nucleic acid molecules is immobilized to the support using an adaptor.

In some embodiments, the detectable moiety is part of the terminating subunit. In some embodiments, the terminating subunit is part of the detectable moiety. In some embodiments, the detectable moiety is the terminating subunit. In some embodiments, the detector has variable optical magnification.

An additional aspect of the disclosure provides a method for determining a nucleic acid sequence of a target nucleic acid molecule. The method comprises: (a) immobilizing a plurality of nucleic acid molecules to a support, where each of the plurality of nucleic acid molecules exhibits sequence homology to the target nucleic acid molecule, and where the support is operatively coupled to a detector; (b) directing a plurality of nucleotides or nucleotide analogs to the support, where the plurality of nucleotides or nucleotide analogs comprises at least a first subset of nucleotides or nucleotide analogs and a second subset of nucleotides or nucleotide analogs, where (i) the first subset of nucleotides or nucleotide analogs comprises nucleotides or nucleotide analogs that are labeled and terminated, and (ii) the second subset of nucleotides or nucleotide analogs comprises nucleotides or nucleotide analogs that are unlabeled and unterminated; (c) subjecting the plurality of nucleic acid molecules to an incorporation reaction under conditions that are sufficient to incorporate the first subset of nucleotides or nucleotide analogs and the second subset of nucleotides or nucleotide analogs into the plurality of nucleic acid molecules, where during incorporation, a given nucleotide or nucleotide analog from the first subset of nucleotides or nucleotides analogs is incorporated into a given nucleic acid molecule from the plurality of nucleic acid molecules, which given nucleotide or nucleotide analog is labeled and terminated; and (d) using the detector to detect the given nucleotide or nucleotide analog, thereby determining the nucleic acid sequence of the target nucleic acid molecule.

In some embodiments, a ratio of the first subset of nucleotides or nucleotide analogs to the second subset of nucleotides or nucleotide analogs is less than 50%, less than 10%, less than 1%, less than 0.1%, less than 0.01%, less than 0.001% or less than 0.0001%.

In some embodiments, the target nucleic acid molecule is a deoxyribonucleic acid molecule. In some embodiments, the method further comprises subjecting the target nucleic acid molecule to nucleic acid amplification (e.g., polymerase chain reaction, emulsion-based amplification, bridge amplification) to generate the plurality nucleic acid molecules. In some embodiments, the target nucleic acid molecule is a ribonucleic acid molecule. In such embodiments, the method may further comprise subjecting the target nucleic acid molecule to reverse transcription to generate the plurality nucleic acid molecules.

In some embodiments, the support is a solid support. In some embodiments, the support is a biological support, non-biological support, organic support, inorganic support, or any combination thereof. In some embodiments, the first subset of nucleotides or nucleotide analogs comprises nucleotides or nucleotide analogs that are each labeled with a detectable moiety, such as an optically detectable moiety (e.g., a fluorophore). In some embodiments, the detectable moiety is an acceptor or a donor. In some embodiments, nucleotides or nucleotide analogs of the first subset are each terminated with a terminating subunit. In some embodiments, the terminating subunit is the detectable moiety.

In some embodiments, the given nucleotide or nucleotide analog from the first subset of nucleotides or nucleotide analogs, after incorporation into the given nucleic acid molecule, reduces a rate of incorporation of a next nucleotide or nucleotide analog into the given nucleic acid molecule.

In some embodiments, the method further comprises cleaving, bleaching, quenching or disabling the detectable moiety. In some embodiments, the detectable moiety is cleaved, bleached, quenched or disabled subsequent to detecting the detectable moiety from the given nucleotide or nucleotide analog. In some embodiments, the first subset of nucleotides or nucleotide analogs comprises nucleotides or nucleotide analogs that are each terminated with a terminating subunit. In some embodiments, nucleotides or nucleotide analogs of the first subset are each labeled with a detectable moiety. In some embodiments, the detectable moiety is at least a portion of the terminating subunit.

In some embodiments, (b)-(d) are repeated, in some cases without cleaving a terminating subunit of the given nucleotide or nucleotide analog. In some embodiments, (d) comprises spectrally shifting an excitation wavelength of an optically detectable moiety. In some embodiments, in (d), the given nucleotide or nucleotide analog is detected via Förster resonance energy transfer (FRET). In some embodiments, the plurality of nucleotides or nucleotide analogs include bases of a first type, and where (b)-(d) are repeated with an additional plurality of nucleotides or nucleotide analogs including bases of a second type different than the first type.

In some embodiments, the plurality of nucleotides or nucleotide analogs is incorporated using a nucleic acid polymerizing enzyme, such as with a deoxyribonucleic acid polymerase. In some embodiments, the deoxyribonucleic acid polymerase is phi-29 or a variant thereof. In some embodiments, the given nucleotide or nucleotide analog is detected while incorporating the given nucleotide or nucleotide analog into the given nucleic acid molecule. In some embodiments, the given nucleotide or nucleotide analog is detected subsequent to incorporating the given nucleotide or nucleotide analog into the given nucleic acid molecule.

In some embodiments, the support is in optical communication with the detector. In some embodiments, the support has a plurality of independently addressable locations. In some embodiments, the plurality nucleic acid molecules is immobilized to the support at a given independently addressable location of the plurality of independently addressable locations. In some embodiments, each of the plurality nucleic acid molecules is immobilized to the support using an adaptor.

In some embodiments, (d) comprises determining a first signal indicative of incorporation of the given nucleotide or nucleotide analog into the given nucleic acid molecule, comparing the first signal indicative of incorporation of the given nucleotide or nucleotide analog to a second signal indicative of incorporation of a previous nucleotide or nucleotide analog incorporated before the given nucleotide or nucleotide analog in the given nucleic acid molecule, and comparing a difference in the first signal and the second signal to one or more predetermined signals for the given nucleotide or nucleotide analog to determine the nucleic acid sequence of the target nucleic acid molecule.

Another aspect of the disclosure provides a method for sequencing a target nucleic acid molecule. The method comprises: (a) subjecting a plurality of nucleic acid molecules exhibiting sequence homology to the target nucleic acid molecule to at most 4000 cycles a nucleic acid extension reaction while measuring detectable signals from the plurality of nucleic acid molecules, which detectable signals correspond to individual nucleotides or nucleotide analogs incorporated into the plurality of nucleic acid molecules during the nucleic acid extension reaction; and (b) using the detectable signals to generate a sequence of the target nucleic acid molecule at a length of at least about 500 bases and an accuracy of at least about 97%. In some embodiments, the accuracy is at least 97% without resequencing.

In some embodiments, the length is at least about 600 bases, at least about 700 bases, at least about 800 bases, at least about 900 bases or at least about 1000 bases. In some embodiments, the accuracy is at least about 98% or at least about 99%. In some embodiments, the sequence is generated in the absence of read alignment.

An additional aspect of the disclosure provides a system for determining a nucleic acid sequence of a target nucleic acid molecule. The system comprises: a support for immobilizing a plurality nucleic acid molecules, where each of the plurality of nucleic acid molecules exhibits sequence homology to the target nucleic acid molecule, and where the support is operatively coupled to a detector; and a controller comprising one or more computer processors that are individually or collectively programmed to: (a) direct a plurality of nucleotides or nucleotide analogs to the support, which plurality of nucleotides or nucleotide analogs comprises at least a first subset of nucleotides or nucleotide analogs and a second subset of nucleotides or nucleotide analogs, where (i) each of the first subset of nucleotides or nucleotide analogs comprises a detectable moiety and a terminating subunit, and (ii) none of the second subset of nucleotides or nucleotide analogs comprises the detectable moiety and the terminating subunit, where the plurality of nucleotides or nucleotide analogs are incorporated into the plurality nucleic acid molecules, where during incorporation, a given nucleotide or nucleotide analog from the first subset of nucleotides or nucleotides analogs is incorporated into a given nucleic acid molecule from the plurality of nucleic acid molecules, which given nucleotide or nucleotide analog comprises the detectable moiety and the terminating subunit; and (b) use the detector to detect the detectable moiety from the given nucleotide or nucleotide analog, thereby determining the nucleic acid sequence of the target nucleic acid molecule.

In some embodiments, the support is part of a chip. In some embodiments, the controller is part of the chip. In some embodiments, the system further comprises the detector. In some embodiments, the support is in optical communication with the detector. In some embodiments, the support has a plurality of independently addressable locations.

Another aspect of the disclosure provides a system for determining a nucleic acid sequence of a target nucleic acid molecule. The system comprises: a support for immobilizing a plurality nucleic acid molecules, where each of the plurality of nucleic acid molecules exhibits sequence homology to the target nucleic acid molecule, and where the support is operatively coupled to a detector; and a controller comprising one or more computer processors that are individually or collectively programmed to: (a) direct a plurality of nucleotides or nucleotide analogs to the support, where the plurality of nucleotides or nucleotide analogs comprises at least a first subset of nucleotides or nucleotide analogs and a second subset of nucleotides or nucleotide analogs, where (i) the first subset of nucleotides or nucleotide analogs comprises nucleotides or nucleotide analogs that are labeled and terminated, and (ii) the second subset of nucleotides or nucleotide analogs comprises nucleotides or nucleotide analogs that are unlabeled and unterminated; (b) subject the plurality of nucleic acid molecules to an incorporation reaction under conditions that are sufficient to incorporate the first subset of nucleotides or nucleotide analogs and the second subset of nucleotides or nucleotide analogs into the plurality of nucleic acid molecules, where during incorporation, a given nucleotide or nucleotide analog from the first subset of nucleotides or nucleotides analogs is incorporated into a given nucleic acid molecule from the plurality of nucleic acid molecules, which given nucleotide or nucleotide analog is labeled and terminated; and (c) use the detector to detect the given nucleotide or nucleotide analog, thereby determining the nucleic acid sequence of the target nucleic acid molecule.

In some embodiments, the support is part of a chip. In some embodiments, the controller is part of the chip. In some embodiments, the system further comprises the detector. In some embodiments, the support is in optical communication with the detector. In some embodiments, the support has a plurality of independently addressable locations.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1G schematically illustrates an example of a system and method for sequencing a nucleic acid molecule;

FIG. 2 schematically illustrates a computer control system that is programmed or otherwise configured to implement methods provided herein;

FIG. 3 schematically illustrates an example method for sequencing a nucleic acid molecule; and

FIGS. 4A-4C graphically depict excitation spectra used in an example detection method described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The terms “amplifying, “amplification,” and “nucleic acid amplification” are used interchangeably and generally refer to generating one or more copies of a nucleic acid. For example, “amplification” of DNA generally refers to generating one or more copies of a DNA molecule. Moreover, amplification of a nucleic acid may be linear, exponential, or a combination thereof. Amplification may be emulsion based or may be non-emulsion based. Amplification may comprise thermal cycling (e.g., one or more heating and cooling cycles). Amplification may be isothermal, such as by conducting amplification at a given temperature or temperature range. Non-limiting examples of nucleic acid amplification methods include reverse transcription, primer extension, polymerase chain reaction (PCR), ligase chain reaction (LCR), helicase-dependent amplification (HDA), asymmetric amplification, rolling circle amplification (RCA), recombinase polymerase amplification (RPA), strand displacement amplification (SDA), and multiple displacement amplification (MDA). Where PCR is used, any form of PCR may be used, with non-limiting examples that include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR and touchdown PCR. Moreover, amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C. C. PNAS, 1989, 86, 4076-4080 and U.S. Pat. Nos. 5,409,811 and 5,674,716, each of which is herein incorporated by reference in its entirety.

The term “detectable moiety” as used herein, generally refers to a moiety that emits a signal that can be detected. In some cases, such a signal may be indicative of incorporation of one or more nucleotides or nucleotide analogs during a primer extension reaction. In some cases, a detectable moiety is coupled to a nucleotide or nucleotide analog, which nucleotide or nucleotide analog may be used in a primer extension reaction. Coupling may be covalent or non-covalent (e.g., via ionic interactions, Van der Waals forces, etc.). Where covalent coupling is implemented, the detectable moiety may be coupled to the nucleotide or nucleotide analog via a linker, with non-limiting examples that include aminopropargyl, aminoethoxypropargyl, polyethylene glycol, polypeptides, fatty acid chains, hydrocarbon chains and disulfide linkages. In some cases, the linker is cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase or protease).

The term “nucleic acid,” or “polynucleotide,” as used herein, generally refers to a molecule comprising one or more nucleic acid subunits, or nucleotides. A nucleic acid may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (P03) groups. A nucleotide can include a nucleobase, a five-carbon sugar (either ribose or deoxyribose), and one or more phosphate groups.

Ribonucleotides are nucleotides in which the sugar is ribose. Deoxyribonucleotides are nucleotides in which the sugar is deoxyribose. A nucleotide can be a nucleoside monophosphate or a nucleoside polyphosphate. A nucleotide can be a deoxyribonucleoside polyphosphate, such as, e.g., a deoxynucleoside triphosphate (dNTP), which can be selected from deoxyadenosine triphosphate (dATP), deoxycytidine triphosphate (dCTP), deoxyguanosine triphosphate (dGTP), deoxyuridine triphosphate (dUTP) and deoxythymidine triphosphate (dTTP), that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a nucleic acid is deoxyribonucleic acid (DNA), ribonucleic acid (RNA), or derivatives or variants thereof. A nucleic acid may be single-stranded or double stranded. In some cases, a nucleic acid molecule is circular.

The terms “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or ribonucleotides (RNA), or analogs thereof. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, or 50 kb. An oligonucleotide is typically composed of a specific sequence of nucleotide bases: adenine (A); cytosine (C); guanine (G); thymine (T); and uracil (U). Thus, the term “oligonucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Oligonucleotides may include one or more nonstandard nucleotide(s), nucleotide analog(s) and/or modified nucleotides.

Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thio triphosphate and beta-thio triphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP), aminohexhylacrylamide-dCTP (aha-dCTP), and propargylamine to allow covalent attachment of amine reactive moieties, such as N-hydroxy succinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. Nat. Chem. Biol. 2012 July; 8(7):612-4, which is herein incorporated by reference for all purposes.

The term “polymerase,” as used herein, generally refers to any enzyme capable of catalyzing a polymerization reaction. Examples of polymerases include, without limitation, a nucleic acid polymerase. The polymerase can be naturally occurring or synthetic. In some cases, a polymerase may have relatively high processivity. Processivity may be increased by adding an affinity tag, such as a single stranded DNA binding domain. An example polymerase is a phi29 (D29) polymerase or derivative thereof. A polymerase can be a polymerization enzyme. In some cases, a transcriptase or a ligase is used (i.e., enzymes which catalyze the formation of a bond). Examples of polymerases include a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Tth polymerase, Pfutubo polymerase, Pyrobest polymerase, Pwo polymerase, KOD polymerase, Bst polymerase, Bsu polymerase, Therminator polymerase, Sac polymerase, Klenow fragment, polymerase with 3′ to 5′ exonuclease activity, and variants, modified products and derivatives thereof. In some embodiments, the polymerase is a single subunit polymerase. The polymerase can have high processivity, namely the capability of the polymerase to consecutively incorporate nucleotides into a nucleic acid template without releasing the nucleic acid template. In some cases, a polymerase is a polymerase modified to accept dideoxynucleotide triphosphates, such as for example, Taq polymerase having a 667Y mutation (see e.g., Tabor et al, PNAS, 1995, 92, 6339-6343, which is herein incorporated by reference in its entirety for all purposes). In some cases, a polymerase is a polymerase having a modified nucleotide binding, which may be useful for nucleic acid sequencing, with non-limiting examples that include ThermoSequenas polymerase (GE Life Sciences), AmpliTaq FS (ThermoFisher) polymerase and Sequencing Pol polymerase (Jena Bioscience). In some cases, the polymerase is genetically engineered to have discrimination against dideoxynucleotides, such, as for example, Sequenase DNA polymerase (ThermoFisher).

The terms “adaptor(s)”, “adapter(s)” and tag(s)” may be used synonymously. An adaptor or tag can be coupled to a polynucleotide sequence to be tagged by any approach, such as, for example, ligation or hybridization. An adaptor or tag can increase processivity of the polynucleotide sequence.

The term “subject,” as used herein, generally refers to an individual having a biological sample that is undergoing processing or analysis. A subject can be an animal or plant. A subject can be a microbe or a virus. The subject can be a mammal, such as a human, dog, cat, horse, pig or rodent, an avian, or other organism. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer or cervical cancer) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “sample,” as used herein, generally refers to a biological sample. Examples of biological samples include tissues, cells, nucleic acid molecules, amino acids, polypeptides, proteins, carbohydrates, fats, or viruses. In an example, a biological sample is a nucleic acid sample including one or more nucleic acid molecules. The nucleic acid molecules may be cell-free or cell-free nucleic acid molecules, such as cell free DNA or cell free RNA. The nucleic acid molecules may be derived from a variety of sources including human, mammal, non-human mammal, ape, monkey, chimpanzee, reptilian, amphibian, or avian, sources. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject), or may be derived from tissue of the subject itself.

The term “adjacent to,’ as used herein, generally means next to, in proximity to, or in sensing, optical, or electronic vicinity (or proximity) of. For example, a first object adjacent to a second object can be in contact with the second object, or may not be in contact with the second object but may be in proximity to the second object. In some examples, a first object adjacent to a second object is within about 0 micrometers (“microns”), 0.001 microns, 0.01 microns, 0.1 microns, 0.2 microns, 0.3 microns, 0.4 microns, 0.5 microns, 1 micron, 2 microns, 3 microns, 4 microns, 5 microns, 10 microns, or 100 microns of the second object.

### Methods for Nucleic Acid Sequencing

In an aspect of the present disclosure, a method for determining a nucleic acid sequence of a target nucleic acid molecule comprises providing a plurality nucleic acid molecules immobilized to a support. The support can be operatively coupled to a detector. Each of the plurality of nucleic acid molecules may exhibit sequence homology to the target nucleic acid molecule.

Next, a plurality of nucleotides or nucleotide analogs may be directed to the support. The plurality of nucleotides or nucleotide analogs may comprise at least a first subset of nucleotides or nucleotide analogs and a second subset of nucleotides or nucleotide analogs. Each of the first subset of nucleotides or nucleotide analogs may comprise a detectable moiety and a terminating subunit. In some cases, none of the second subset of nucleotides or nucleotide analogs comprises the detectable moiety and the terminating subunit.

Next, the plurality of nucleotides or nucleotide analogs comprising the first subset of nucleotides or nucleotide analogs and the second subset of nucleotides or nucleotide analogs may be incorporated into the plurality of nucleic acid molecules. During incorporation, a given nucleotide or nucleotide analog from the first subset of nucleotides or nucleotides analogs may be incorporated into a given nucleic acid molecule from the plurality of nucleic acid molecules. The given nucleotide or nucleotide analog may comprise the detectable moiety and the terminating subunit.

The detector may be used to detect the detectable moiety from the given nucleotide or nucleotide analog. This may be used to determine the nucleic acid sequence of the target nucleic acid molecule. Moreover, a detector may implement one or more detection methods, with a detector described by the detection method(s) it implements. For example, a detector that implements an optical detection method can be considered an optical detector. Non-limiting examples of detection methods (e.g., implemented with corresponding detectors) include optical detection, spectroscopic detection and electronic detection. Optical detection methods include fluorimetry, ultraviolet-visible (UV-vis) light absorbance and microscopy (e.g., via photographs or video, such as via a CCD camera). In some cases, optical detection may include the use of a waveguide. In some cases, spectroscopic detection methods include mass spectrometry, nuclear magnetic resonance (NMR) spectroscopy, and infrared spectroscopy. An example of electronic detection is the detection of charge or changes in charge via, for example, a field effect transistor (FET), such as an ion sensitive FET or chemFET.

A ratio of the first subset of nucleotides or nucleotide analogs to the second subset of nucleotides or nucleotide analogs may be at most about 50%, 40%, 30%, 20%, 10%, 5%, 1%, 0.5%, 0.4%, 0.3%, 0.2%, 0.1%, 0.01%, 0.001%, 0.0001%, 0.00001%, 0.000001%, 0.0000001%, or less. In some cases, a ratio of the first subset of nucleotides or nucleotide analogs to the second subset of nucleotides or nucleotide analogs may be at least about 0.0000001%, 0.000001%, 0.00001%, 0.0001%, 0.001%, 0.01%, 0.1%, 0.2%, 0.3%, 0.4%, 0.5%, 1%, 5%, 10%, 20%, 30%, 40%, 50%, or more. In some cases, a ratio of the first subset of nucleotides or nucleotide analogs to the second subset of nucleotides or nucleotide analogs may be within a range from about 0.0001% to about 1%, from about 0.0001% to about 10%, from about 1% to about 10%, from about 0.0001% to about 50%, from about 1% to about 50%, from about 10% to about 50%, or any range overlapping or non-overlapping with the above.

The ratio may be about the reciprocal of the remaining length to be read. The ratio may be the reciprocal of the anticipated remaining length to be read. For example, if the remaining length to be read or the anticipated remaining length to be read is 100, the ratio of the first subset of nucleotides or nucleotide analogs to the second subset of nucleotides or nucleotide analogs may be about equal to 1/100, or 0.01. After a base pair is read, the remaining length to be read or the anticipated remaining length to be read is about 99, thus making the subsequent ratio of the first subset of nucleotides or nucleotide analogs to the second subset of nucleotides or nucleotide analogs may be about equal to 1/99. Therefore the ratio of the first subset of nucleotides or nucleotide analogs to the second subset of nucleotides or nucleotide analogs over sequential reads may be modeled as approximately 1/x, where x is the length of the sequence read corresponding to the nucleic acid remaining to be sequenced.

The functional relationship between the ratio of the first subset of nucleotides or nucleotide analogs to the second subset of nucleotides or nucleotide analogs and the reads remaining to be sequenced may also take the form 1/(x+c), where x is the length of the sequence read corresponding to the nucleic acid remaining to be sequenced and c is a corrective factor. When c is positive it acts as a dilution factor. When c is negative it acts as a concentrating factor. When c is a concentrating factor the magnitude of c may be less than the value of x. The corrective factor, c, may take on integer and non-integer values. The corrective factor, c, may be used to fix a signal-to-noise ratio, to maintain the signal-to-noise ratio above a minimum threshold, and/or to target individual nucleotides, nucleotide analogs, read lengths, and/or read positions. Other functions relating the first subset of nucleotides or nucleotide analogs to the second subset of nucleotides or nucleotide analogs may be contemplated including 1/(bx), 1/(x^{b}), 1/(b^{x}), 1/(e^{bx}), 1/(be^{x}), etc., where x is the length of sequence read corresponding to the nucleic acid remaining to be sequenced and b is a corrective parameter adjusting the ratio of the first subset of nucleotides or nucleotide analogs to the second subset of nucleotides or nucleotide analogs.

The functional relationship between the ratio of the first subset of nucleotides or nucleotide analogs to the second subset of nucleotides or nucleotide analogs and the reads that have been sequenced may also take the form 1/(y+f), where y is the length of the sequence read corresponding to the nucleic acid that has been sequenced and f is a corrective factor. When f is positive it acts as a dilution factor. When f is negative it acts as a concentrating factor. When f is a concentrating factor the magnitude off may be less than the value off. The corrective factor, f, may take on integer and non-integer values. The corrective factor, f, may be used to fix a signal-to-noise ratio, to maintain the signal-to-noise ratio above a minimum threshold, and/or to target individual nucleotides, nucleotide analogs, read lengths, and/or read positions. Other functions relating the first subset of nucleotides or nucleotide analogs to the second subset of nucleotides or nucleotide analogs may be contemplated including 1/(gy), 1/(y^{g}), 1/(g^{y}), 1/(e^{gy}), 1/(ge^{y}), etc., where y is the length of sequence read corresponding to the nucleic acid that has been sequenced and g is a corrective parameter adjusting the ratio of the first subset of nucleotides or nucleotide analogs to the second subset of nucleotides or nucleotide analogs.

Functions relating the first subset of nucleotides or nucleotide analogs to the second subset of nucleotides or nucleotide analogs as a function of the length of sequence read corresponding to the nucleic acid remaining to be sequenced as described herein and functions relating the first subset of nucleotides or nucleotide analogs to the second subset of nucleotides or nucleotide analogs as a function of the length of sequence read corresponding to the nucleic acid that has been sequenced as described herein may be combined in any manner, along with their respective corrective factors.

The ratio of the first subset of nucleotides or nucleotide analogs to the second subset of nucleotide analogs may not be the same as the ratio of modified nucleotides or nucleotide analogs (such as those with detectable moieties, those with terminating subunits, or those with detectable moieties and terminating subunits) incorporated or anticipated to be incorporated.

The target nucleic acid molecule may be a deoxyribonucleic acid (DNA) molecule. As an alternative or in addition, the target nucleic acid molecule may be a ribonucleic acid molecule (RNA), such as mRNA. The target nucleic acid molecule may originate from a cell.

In some situations, the target nucleic acid molecule is subjected to nucleic acid amplification to generate the plurality nucleic acid molecules. The nucleic acid amplification may be polymerase chain reaction (PCR) or isothermal amplification. The nucleic acid amplification may be emulsion-based amplification. The nucleic acid amplification may be bridge amplification. The target nucleic acid molecule may be subjected to reverse transcription to generate the plurality of nucleic acid molecules.

The support may be a solid support such as a slide, a bead, a resin, a chip, an array, a matrix, a membrane, a nanopore, or a gel. The solid support may, for example, be a bead on a flat substrate (such as glass, plastic, silicon, etc.) or a bead within a well of a substrate. The substrate may have surface properties, such as textures, patterns, microstructures coatings, surfactants, or any combination thereof to retain the bead at a desired location (such as in a position to be in operative communication with a detector). The detector of bead-based supports may be configured to maintain substantially the same read rate independent of the size of the bead. The support may be a flow cell or an open substrate. Furthermore, the support may comprise a biological support, a non-biological support, an organic support, an inorganic support, or any combination thereof. The support may be in optical communication with the detector, may be physically in contact with the detector, may be in proximity of the detector, may be separated from the detector by a distance, or any combination thereof. The support may have a plurality of independently addressable locations. The nucleic acid molecules may be immobilized to the support at a given independently addressable location of the plurality of independently addressable locations. Immobilization of each of the plurality of nucleic acid molecules to the support may be aided by the use of an adaptor. The support may be optically coupled to the detector.

The detectable moiety may be optically detectable. Detectable moieties include but are not limited to one or more radioisotopes, one or more fluorescent molecules (e.g., a fluorescent label or a fluorophore, e.g., a coumarin, resorufin, xanthene, benzoxanthene, cyanine, xanthine, carbopyronine, or bodipy analog), one or more cheminescent agents, one or more luminescent agents, one or more colorimetric agents, one or more enzyme-substrate labels, one or more quantum dots or a colloidal quantum dots (QDs) (e.g., a QDOT® nanocrystal, Life Technologies, Carlsbad, Calif), or one or more epitopes or binding molecules (e.g., a ligand), or any combination thereof. The detectable moiety may be an acceptor or a donor. The detectable moiety may be detectable via Forster resonance energy transfer (FRET). The nucleotide or nucleotide analog, the detectable moiety, the terminating subunit, or any combination thereof may individually or collective comprise one or more biotin molecules and one or more streptavidin molecules.

In some embodiments, an optically detectable agent comprises a dye. Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyllaminol fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, Atto dyes, Abberior dyes, Dyomics dyes, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores. In some cases, a dye may be a polymeric dye or a dye comprising a polymeric species. Additional examples of such dyes are described in U.S. Pat. Nos. 9,547,008; 9,383,353; 9,139,869; 8,969,509; 8,802,450; 8,575,303; 8,455,613; 8,362,193; 8,354,239 and 8,158,44, each of which is herein incorporated by reference in its entirety.

The terminating subunit may be part of the detectable moiety, the detectable moiety may be part of the terminating subunit, and/or the detectable moiety may be the terminating subunit. A nucleotide or nucleotide analog with a terminating subunit can be a terminator. A terminator can be any nucleotide or nucleotide analog that prevents, or causes any reduction in rate of, a reaction (e.g., incorporation reaction) of the next nucleotide or nucleotide analog in the sequence. The related terms of “terminate,” “terminated,” and “terminating,” as used herein, may refer to the prevention of, or the reduction in rate of, a reaction of the next nucleotide or nucleotide analog in the sequence and/or contribution thereto. For example, the next nucleotide or nucleotide analog can have a rate (of incorporation) of zero or non-zero. The reduction in rate can be of the same molecule, of a different nucleotide or nucleotide analog with or without a detectable moiety. Detection of a reduction in rate (of incorporation) between different nucleotides or nucleotide analogs can be particularly beneficial for homopolymer determination. The terminator can be a chain terminator. The terminator can be a reversible terminator, for example, reversible by cleavage of part or whole of the terminating subunit. The terminator can be a true terminator, for example, which after incorporation prevents a reaction of the next nucleotide or nucleotide analog in the sequence, wherein the next nucleotide or nucleotide analog is any substrate (e.g., with or without detectable moieties).

The terminator can be a virtual terminator, for example, which after incorporation prevents, or causes any reduction in rate of, a reaction of the next nucleotide or nucleotide analog in the sequence. In some cases, a virtual terminator can be referred to as an imperfect virtual terminator, or an attenuator, for example, if after incorporation it causes a reduction in rate of a reaction of the next nucleotide or nucleotide analog in the sequence. In some instances, a virtual terminator may reduce the rate of reaction (e.g., incorporation reaction) of the next substrate (e.g., nucleotide or nucleotide analog) by different degrees depending on the type of the next substrate. In some instances, different virtual terminators (e.g., comprising different inhibitors) may reduce the rate of reaction of the next substrate of a particular type (e.g., particular type of base) by different degrees. Signals indicative of incorporation of a virtual terminator and/or signals indicative of incorporation of the next nucleotide or nucleotide analog in the sequence may be detected. In some cases, the signals can be determined at different time points and/or in real-time. In some cases, such time data may be indicative of a rate of reaction and/or a reduction in rate of reaction caused by incorporation of a virtual terminator. One or more signals indicative of incorporation of a virtual terminator, one or more signals indicative of a substrate incorporated after the virtual terminator (e.g., next substrate after the virtual terminator), and other such contextual data, associated with or caused by the virtual terminator may be predetermined and stored in memory, such as in one or more databases. The one or more databases can comprise one or more of a chart, table, graph, index, hash database, and/or other data structures. Such predetermined signals may be based at least in part on empirical data, theoretical data, statistical analysis, and/or a combination thereof. In some cases, computations comparing the signals indicative of incorporation of two different substrates (collectively or individually) can be performed, such as to determine a mathematical difference, sum, ratio, percentage, product, mean, or other computed value and stored in memory. Computations may be linear or non-linear comparisons. Various computations may be made by one or more processors, microprocessors, controllers, microcontrollers, and/or other computer systems described elsewhere herein.

The difference between a first signal (indicative of incorporation of a first nucleotide or nucleotide analog) and a second signal (indicative of incorporation of a next nucleotide or nucleotide analog) may be readily measured by detection methods described elsewhere herein. For example, the detectable moieties of a plurality of nucleotides or nucleotide analogs may be detected at different time points during or subsequent to incorporation of the nucleotides or nucleotide analogs to determine the incorporation of each nucleotide or nucleotide analog. In some cases, detectable signals received at different times can be compared. This time data can be compared to the predetermined data of detectable signals (and/or rates of reactions) for the terminator to determine the sequence. In some cases, a signal indicative of incorporation of a nucleotide or nucleotide analog can be measured in real-time. Real-time can include a response time of less than 1 second, tenths of a second, hundredths of a second, a millisecond, or less. All of the detections and measurements by a detector, such as those described above or further below, are capable of happening in real-time. All of the determinations made by a computer system, including one or more computations and/or comparisons, may be capable of happening in real-time.

The plurality of nucleotides or nucleotide analogs may include deoxynucleotides or dideoxynucleotides, including but not limited to deoxyadenosine triphosphate (dATP), 2′,3′-dideoxyadenosine-5′-triphosphate (ddATP), deoxyguanosine triphosphate (dGTP), 2′,3′-dideoxyguanosine-5′-triphosphate (ddGTP), deoxycytidine triphosphate (dCTP), 2′,3′-dideoxycytidine-5′-triphosphate (ddCTP), deoxythymidine triphosphate (dTTP), 2′,3′-dideoxythymidine-5′-triphosphate (ddTTP), deoxyuridine triphosphate (dUTP), 2′,3′-dideoxyuridine-5′-triphosphate (ddUTP), or a variant thereof. Alternatively or in addition, the plurality of nucleotides or nucleotide analogs may include virtual terminators. A virtual terminator may possess a free 3′ hydroxyl but be capable of blocking, or reducing the rate of, a next nucleotide or nucleotide analog from incorporating. For example, a virtual terminator may comprise a free 3′ hydroxyl (—OH) maintaining natural interactions at the polymerase active site, a base modified with a propargylamine connected to a cleavable linker, and a detectable moiety tethered to an inhibitor, attached via the cleavable linker. The inhibitor may or may not comprise a phosphate group (e.g., monophosphate, biphosphate, etc.). In some instances, the virtual terminators can include 2-nitrobenzyl-modified thymidine analogs based on 5-hydroxymethyl-2′-deoxyuridine-5′-triphosphate (HOMedUTP) (e.g., 5-(2-nitrobenzyloxy)methyl-dUTP analogs), 7-deaza-7-hydroxymehtyl-2′-deoxyadenosine-5′-triphosphate (C⁷-HOMedATP), 5-hydroxymethyl-2′-deoxycytidine-5′-triphosphate (HOMedCTP), and 7-deaza-7-hydroxymethyl-2′-deoxyguanosine-5′-triphosphate (C⁷-HOMedGTP). The 2-nitrobenzyl group can be photocleavable (e.g., via UV light).

The plurality of nucleotides or nucleotide analogs may include bases of the same type.

The operations of directing the plurality of nucleotides or nucleotide analogs (themselves comprising at least a first subset of nucleotides or nucleotide analogs that are labeled and terminated and a second subset of nucleotides or nucleotide analogs that are unlabeled and unterminated) to the support, subjecting the plurality of nucleic acid molecules to an incorporation reaction under conditions that are sufficient to incorporate the first subset of nucleotides or nucleotide analogs and the second subset of nucleotides or nucleotide analogs into the plurality of nucleic acid molecules, and using the detector to detect the given nucleotide or nucleotide analog to determine the nucleic acid sequence of the target nucleic acid molecule may be repeated. This sequence may be repeated any number of times from zero to the number needed to determine the nucleic acid sequence. Beneficially, this sequence may be repeated without cleaving a terminating subunit of the given nucleotide or nucleotide analog. The plurality of nucleotides or nucleotide analogs may include bases of a first type and each repetition of the aforementioned sequence of directing the plurality of nucleotides or nucleotide analogs to the support, subjecting the plurality of nucleic acid molecule to an incorporation reaction, and using the detector to detect the given nucleotide or nucleotide analogs may comprise an additional plurality of nucleotides or nucleotide analogs including bases of a second type different than the first type. In some cases, the additional plurality of nucleotides or nucleotide analogs may include a third subset of nucleotides or nucleotide analogs, each of which has an additional detectable moiety different than the detectable moiety of the first subset of nucleotides or nucleotide analogs.

When directing the plurality of nucleotides or nucleotide analogs comprising at least a first subset of nucleotides or nucleotide analogs that are labeled and terminated and a second subset of nucleotides or nucleotide analogs that are unlabeled and unterminated to the support, the first subset of nucleotides or nucleotide analogs and the second subset of nucleotides or nucleotide analogs may be delivered simultaneously. When directing the plurality of nucleotides or nucleotide analogs to the support, the first subset of nucleotides or nucleotide analogs may be delivered before the second subset of nucleotides or nucleotide analogs to allow for the possibly slower incorporation of modified nucleotides or nucleotide analogs. When directing the plurality of nucleotides or nucleotide analogs to the support, the first subset of nucleotides or nucleotide analogs may be delivered after the second subset of nucleotides or nucleotide analogs. When directing the plurality of nucleotides or nucleotide analogs to the support, the first subset of nucleotides or nucleotide analogs may be delivered in the absence of the second subset of nucleotides or nucleotide analogs or the second subset of nucleotides or nucleotide analogs may be delivered in the absence of the first subset of nucleotides or nucleotide analogs. When directing the plurality of nucleotides or nucleotide analogs to the support, the first subset of nucleotides or nucleotide analogs and the second subset of nucleotides or nucleotide analogs may be delivered sequentially such that the first subset of nucleotides or nucleotide analogs is delivered before the second subset of nucleotides or nucleotide analogs.

In some cases, when the plurality of nucleotides or nucleotide analogs are virtual terminators, each of the nucleotides or nucleotide analogs can be labeled, and signals indicative of incorporation of each of the nucleotides or nucleotide analogs, individually or collectively, can be detected. The signals can be detected at different time points, such as during or subsequent to incorporation of each nucleotide or nucleotide analog. In some cases, the difference in signals, if any, between consecutive nucleotides or nucleotide analogs can be measured and compared to determine incorporation of a nucleotide or nucleotide analog. In some cases, based at least in part on a comparison between the time of incorporation, or time of substantial incorporation such that a signal is detected, of two consecutive nucleotides or nucleotide analogs, a reduction in reaction rate (e.g., caused by a virtual terminator) can be determined and compared to predetermined reaction rates for the terminator to determine the sequence. For example, the reduction in reaction rate can be computed as a difference or ratio between two or more rates.

For those optically detectable moieties, the nucleotide or nucleotide analog (e.g., deoxynucleotides, dideoxynucleotides) of each base type may have the same dye and thus may be excited by and/or transmit the same wavelength of light. The optically detectable moieties may comprise a dye of at least one color. The color of the dye—the color of the light that significantly excites, detects, or is transmitted by the dye, detectable moiety, the terminating subunit, or both—may be on the visible light spectrum with a wavelength falling with the range from about 390 nanometers to about 700 nanometers, may be within the infrared spectrum with the range from about 700 nanometers to about 1 millimeter, may be within the ultraviolet spectrum from about 10 nanometers to about 390 nanometers, or it may be any combination of the aforementioned ranges.

The optically detectable moieties may comprise dyes at least two colors. As a non-limiting example, the optically detectable moieties associated with purines may receive a first dye with an associated first color and the optically detectable moieties associated with pyrimidines may receive a second dye with an associated second color. As another non-limiting example, the optically detectable moieties of a first set of complementary bases (e.g., adenine and thymine) may receive a first dye with an associated first color and the optically detectable moieties of a second set of complementary bases (e.g., guanine and cytosine) may receive a second dye with an associated second color. The colors associated with the dyes may correspond to dyes that are excited by light of different wavelengths or may transmit light of different wavelengths. Such excitation may be by way of an excitation source, such as a laser. The excitation source may be provided continuously or intermittently (e.g., pulses of excitation).

The optically detectable moiety may comprise dyes of at least three colors.

The optically detectable moieties may comprises a number of dyes equal to the number of base types, such that each base (e.g., adenine, guanine, cytosine, thymine) has its dye with its own associated color, each color distinct from each other color. The colors associated with the dyes may correspond to dyes that are excited by light of different wavelengths or may transmit light of different wavelengths.

Different dyes may be used as more of the nucleic acid sequence of the target nucleic acid molecule is determined. Such sequence-dependent dye use may be a function of time, signal-to-noise ratio, amount of the sequence that has been read, the amount of sequence remaining to be read, the ratio of the amount of the sequence that has been read to the total anticipated amount of the sequence remaining to be read, or any combination thereof.

The detectable moiety may be detected while incorporating the given nucleotide or nucleotide analog into the given nucleic acid molecule. The detectable moiety may be detected subsequent to incorporating the given nucleotide or nucleotide analog into the given nucleic acid molecule. The detectable moiety may be detected subsequent to incorporating the given nucleotide or nucleotide analog into the given nucleic acid molecule and washing unincorporated nucleotides or nucleotide analogs among the plurality of nucleotides or nucleotide analogs.

Incorporation can be followed by one or more wash cycles. The wash cycles may reduce unincorporated and non-specifically absorbed nucleotides or nucleotide analogs by enzymatic methods. The wash cycles may comprise using various wash buffers. For example, the wash cycles may use alkaline phosphatase, such as shrimp alkaline phosphatase (rSAP)®, FastAP® thermosensitive alkaline phosphatase, calf intestinal alkaline phosphatase (CIAP)®, and other enzymes. In some cases, enzymatic washing can happen in parallel or substantially simultaneously with cleavage.

After a terminator is incorporated, the terminating subunit of the terminator may be cleaved during detection or subsequent to detection. Moreover, the detectable moiety may be cleaved, bleached, quenched or disabled during detection or subsequent to detection of the detectable moiety from the given nucleotide or nucleotide analog. Förster resonance energy transfer may be used to cleave, bleach, quench, or disable the detectable moiety, wherein the binding of an acceptor dye eliminates or spectrally shifts the emission of the emission of the detectable moiety. Cleaving, bleaching, quenching, or disabling the detectable moiety or the terminating subunit may be done one or more times for each cycle of direction, incorporation, and detection of the plurality of nucleotides or nucleotide analogs used in the method to determine the desired nucleic acid sequence. Similarly, cleaving, bleaching, quenching, or disabling the detectable moiety or the terminating subunit may be done one or more times for a subset (e.g., from the tenth cycle to the hundredth cycle out of a total of two hundred cycles) of the repeated cycles of direction, incorporation, and detection of the plurality of nucleotides or nucleotide analogs. In those cases of repeated cycles of direction, incorporation, and detection, cleaving, bleaching, quenching, or disabling may be done at any point, though preferably after detection or before incorporation. In those cases of repeated cycles of direction, incorporation, and detection, cleaving, bleaching, quenching, or disabling may be done with any sort of repetitive pattern. As a non-limiting example, the method may comprise a first cycle wherein cleaving, bleaching, quenching, or disabling is not done, a second cycle where it is, a third where it is not, a fourth where it is, etc. In this way, cleaving, bleaching, quenching, or disabling may be done every other cycle, every third cycle, every fourth cycle, every fifth cycle, every sixth cycle, every seventh cycle, every eighth cycle, every ninth cycle, every tenth cycle, every eleventh cycle, every twelfth cycle, every thirteenth cycle, every fourteenth cycle, every fifteenth cycle, every sixteenth cycle, every seventeenth cycle, every eighteenth cycle, every nineteenth cycle, every twentieth cycle, etc. Cleaving, bleaching, quenching, or disabling may be determined as a function of time, sequence length read, sequence length remaining, signal-to-noise ratio, and/or it may be used to diminish the effects of accumulated detectable moieties. Cleaving, bleaching, quenching, or disabling may be determined as a function of the background signal reached by previous operations, such as when one or more locations exceed a certain level of brightness. Repeated cycles of direction, incorporation, and detection may be done without cleaving the terminating subunits of those nucleotides or nucleotide analogs that are comprised of them. Moreover, any of cleaving, bleaching, quenching or disabling may include the use of stabilizing solution during detection. Such stabilizing solution can minimize or even eliminate photobleaching where desired. To initiate photobleaching, the stabilizing solution can be removed. A stabilizing solution can include any suitable components, including one or more of pyrogallol, ascorbic acid and Trolox. Commercially available stabilizing solutions include SlowFade, ProLong Gold and ProLong Diamond from ThermoFisher.

In one example, various optically detectable moieties and shifts in excitation wavelength with a fluorimeter can be used as an alternative to cleavage of terminating subunits in permitted repeated cycles of direct, incorporation, and detection. In such a strategy, a user may choose an appropriate shift in spectral excitation wavelengths between chosen optically detectable moieties (e.g., a shift approximating the Stokes shift of the preceding optically detectable moieties, such as in the range of 20 nanometers (nm)). The Stokes shift may be at least about 5 nm, 10 nm, 20 nm, 30 nm, 40 nm, 50 nm, or 100 nm. Alternatively, the Stokes shift may be less than or equal to about 100 nm, 50 nm, 40 nm, 30 nm, 20 nm, 10 nm, or 5 nm. Each dye is then excited at its chosen wavelength and its emission measured. The strategy can then be repeated for successively redder dyes (using successfully redder lasers). In this mode, the emission of the each of the optical detectable moieties can be narrowed by shifting the excitation wavelengths, resulting in good resolution of emission spectra and, thus, detection of incorporated terminating subunits having an optical detectable moiety. Moreover, shifting the excitation wavelength with detection can minimize, or even eliminate, detection complications as a result of signal buildup. In some cases a synchronous scan is used, whereby excitation and emission wavelengths are measured at the same time.

As part of such a strategy, each nucleotide in a first set of nucleotides is attached to the same first optically detectable moiety and directed, incorporated and detected for a desired number of cycles. Excitation wavelength for detection is set at the excitation wavelength of the first optically detectable moiety and the readout wavelength is set to its appropriate emission wavelength. In some examples, the number of cycles may be at least about 2 cycles, at least about 5 cycles, at least about 10 cycles, at least about 15 cycles, at least about 20 cycles, at least about 25 cycles, at least about 30 cycles, at least about 35 cycles, at least about 40 cycles, at least about 45 cycles, at least about 50 cycles, at least about 55 cycles, at least about 60 cycles or more. In other examples, the number of cycles may be at most about 60 cycles, at most about 55 cycles, at most about 50 cycles, at most about 45 cycles, at most about 40 cycles, at most about 35 cycles, at most about 30 cycles, at most about 25 cycles, at most about 20 cycles, at most about 15 cycles, at most about 10 cycles, at most about 5 cycles or at most about 2 cycles.

After the desired number of cycles is completed, the process of direction, incorporation and detection is then repeated for another desired number of cycles using a second set of nucleotides. Each nucleotide in the second set is attached to a second optically detectable moiety. The excitation wavelength for detection is then shifted by the desired shift (e.g., the Stokes shift of the first optically detectable moiety) and the emission wavelength set to the appropriate emission wavelength for the second optically detectable moiety. The desired number of cycles, examples of which include those described above for the first optically detectable moiety, may be the same as used for detection of the first optically detectable moiety or may be different. The process of shifting excitation wavelengths followed by detection for a desired number of cycles is then repeated for the desired number of optically detectable moieties, with each subsequent optically detectable moiety being different from the last and having an excitation wavelength (and, thus, emission wavelength) shifted from the prior optically detectable moiety. Any suitable number of optically detectable moieties may be used. For example, the number of optically detectable moieties used in this context may be at least about 2, at least about 3, at least about 4, at least about 5, at least about 6, at least about 7, at least about 8, at least about 9, at least about 10 or more optically detectable moieties. Excitation and detection of emission wavelengths may be completed with any suitable instrument, including a fluorimeter having (or able to be configured to have) multiple lasers (e.g., a BD LSRFortessa instrument, permitting up to five lasers selected from a larger group of lasers).

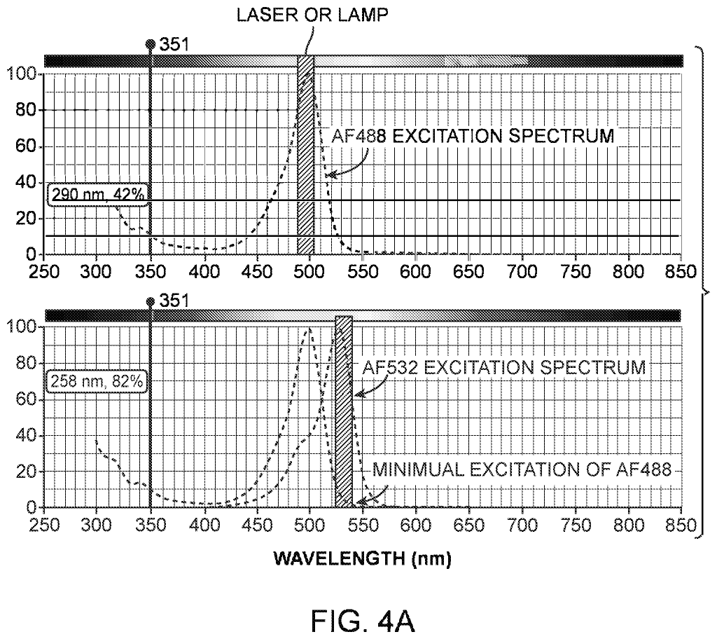

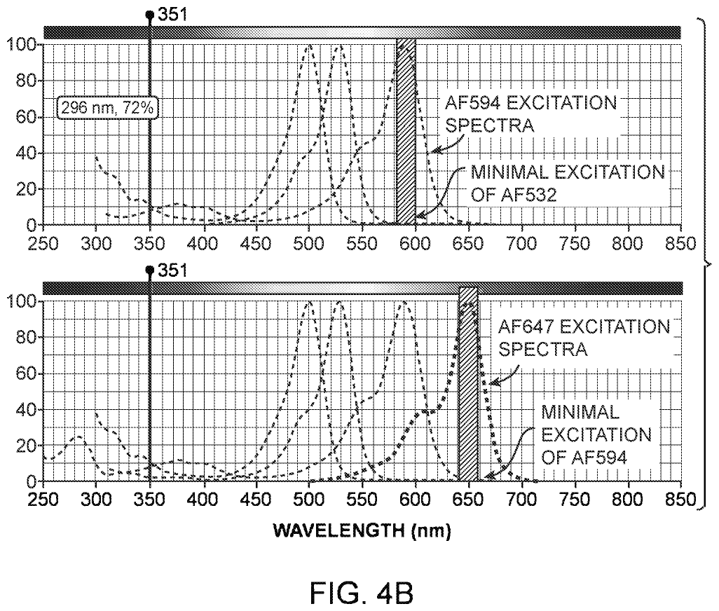

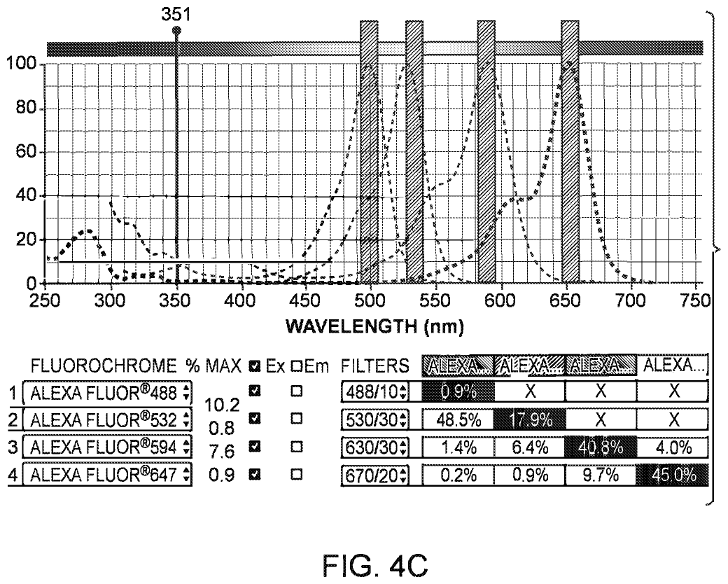

An example of detection using such a strategy is provided below, with relevant excitation spectra shown in FIGS. 4A-4C. In this example, a first set of four nucleotides (e.g. A, T, C, G) each comprising an Alexa Fluor 488 (AF488) dye is directed, incorporated and detected for thirty cycles. As shown in FIG. 4A, the fluorimeter used to detect the AF488 dye is set near the maximum excitation wavelength for the AF488 dye (e.g., at 500 nm) and set to detect in it its emission band (e.g. 540 nm, spectra not shown). After thirty cycles, a second set of the four nucleotides, each comprising an Alexa Fluor 532 (AF532) dye is then directed, incorporated and detected for another thirty cycles. The fluorimeter is then set near maximum excitation wavelength of the AF532 dye (e.g., at 530 nm) and set to detect in its emission band (e.g., 560 nm, spectra not shown). As shown in FIG. 4A, the maximum excitation wavelength of AF532 does not result in significant excitation of the AF488 dye, thus, minimizing any subsequent emission from AF488. After the second set of thirty cycles, a third set of the four nucleotides, each comprising an Alexa Fluor 594 (AF594) dye is then directed, incorporated and detected for another thirty cycles. The fluorimeter is then set near the maximum excitation wavelength of the AF594 dye (e.g., 590 nm) and set to detect in its emission band (e.g., 620 nm, spectra not shown). As shown in FIG. 4B, the maximum excitation wavelength of AF594 does not result in significant excitation of the AF488 or AF532 dyes, thus, minimizing any subsequent emission from these dyes. After the third set of thirty cycles, a fourth set of the four nucleotides, each comprising an Alexa Fluor 647 (AF647) dye is then directed, incorporated and detected for another thirty cycles. The fluorimeter is then set near the maximum excitation wavelength of the AF647 dye (e.g., 650 nm) and set to detect in its emission band (e.g., 680 nm, spectra not shown). As shown in FIG. 4B, the maximum excitation wavelength of AF647 does not result in significant excitation of the AF488, AF532 or AF647 dyes, thus, minimizing any subsequent emission from these dyes. A summary view of the various excitation spectra is shown in FIG. 4C. Using shifts in excitation wavelength, cleavage of terminating subunits is not necessary, as excitation at lower wavelengths is minimized (or even eliminated) with progressively more red measurements.

The plurality of nucleotides or nucleotide analogs may be incorporated using a nucleic acid polymerizing enzyme (e.g., a deoxyribonucleic acid polymerase such as phi-29 or a variant thereof or other polymerase described elsewhere herein).

In another aspect of the present disclosure, a method for determining a nucleic acid sequence of a target nucleic acid molecule comprises immobilizing a plurality of nucleic acid molecules to a support. Each of the plurality of nucleic acid molecules may exhibit sequence homology to the target nucleic acid molecule. The support may be operatively coupled to a detector.

Next, a plurality of nucleotides or nucleotide analogs may be directed to the support. The plurality of nucleotides or nucleotide analogs may comprise at least a first subset of nucleotides or nucleotide analogs and a second subset of nucleotides or nucleotide analogs. The first subset of nucleotides or nucleotide analogs may comprise nucleotides or nucleotide analogs that are labeled and terminated. The second subset of nucleotides or nucleotide analogs may comprise nucleotides or nucleotide analogs that are unlabeled and unterminated.

Next, the plurality of nucleic acid molecules may be subjected to an incorporation reaction under conditions that are sufficient to incorporate the first subset of nucleotides or nucleotide analogs and the second subset of nucleotides or nucleotide analogs into the plurality of nucleic acid molecules. During incorporation, a given nucleotide or nucleotide analog from the first subset of nucleotides or nucleotides analogs (which are labeled and terminated) may be incorporated into a given nucleic acid molecule from the plurality of nucleic acid molecules. The plurality of nucleotides or nucleotide analogs may be incorporated using a nucleic acid polymerizing enzyme. The nucleic acid polymerizing enzyme may be a deoxyribonucleic acid polymerase (such as, for example, phi-29 or a variant thereof or other polymerase described elsewhere herein).

Next, the detector may be used to detect the given nucleotide or nucleotide analog. This may determine the nucleic acid sequence of the target nucleic acid molecule.

A ratio of the first subset of nucleotides or nucleotide analogs to the second subset of nucleotides or nucleotide analogs may be at most about 50%, 40%, 30%, 20%, 10%, 5%, 1%, 0.5%, 0.4%, 0.3%, 0.2%, 0.1%, 0.01%, 0.001%, 0.0001%, 0.00001%, 0.000001%, 0.0000001%, or less. In some cases, a ratio of the first subset of nucleotides or nucleotide analogs to the second subset of nucleotides or nucleotide analogs may be at least about 0.0000001%, 0.000001%, 0.00001%, 0.0001%, 0.001%, 0.01%, 0.1%, 0.2%, 0.3%, 0.4%, 0.5%, 1%, 5%, 10%, 20%, 30%, 40%, 50%, or more. In some cases, a ratio of the first subset of nucleotides or nucleotide analogs to the second subset of nucleotides or nucleotide analogs may be within a range from about 0.0001% to about 1%, from about 0.0001% to about 10%, from about 1% to about 10%, from about 0.0001% to about 50%, from about 1% to about 50%, from about 10% to about 50%, or any range overlapping or non-overlapping with the above.

The ratio may be about the reciprocal of the remaining length to be read. The ratio may be the reciprocal of the anticipated remaining length to be read. For example, if the remaining length to be read or the anticipated remaining length to be read is 100, the ratio of the first subset of nucleotides or nucleotide analogs to the second subset of nucleotides or nucleotide analogs may be about equal to 1/100, or 0.01. Having read a base pair, the remaining length to be read or the anticipated remaining length to be read is about 99, thus making the subsequent ratio of the first subset of nucleotides or nucleotide analogs to the second subset of nucleotides or nucleotide analogs may be about equal to 1/99. Therefore the ratio of the first subset of nucleotides or nucleotide analogs to the second subset of nucleotides or nucleotide analogs over sequential reads may be modeled as approximately 1/x, where x is the length of the sequence read corresponding to the nucleic acid remaining to be sequenced. The functional relationship between the ratio of the first subset of nucleotides or nucleotide analogs to the second subset of nucleotides or nucleotide analogs and the reads remaining to be sequenced may also take the form 1/(x+c), where x is the length of the sequence read corresponding to the nucleic acid remaining to be sequenced and c is a corrective factor. When c is positive it acts as a dilution factor. When c is negative it acts as a concentrating factor. When c is a concentrating factor the magnitude of c may be less than the value of x. The corrective factor, c, may take on integer and non-integer values. The corrective factor, c, may be used to fix a signal-to-noise ratio, to maintain the signal-to-noise ratio above a minimum threshold, and/or to target individual nucleotides, nucleotide analogs, read lengths, and/or read positions. Other functions relating the first subset of nucleotides or nucleotide analogs to the second subset of nucleotides or nucleotide analogs may be contemplated including 1/(bx), 1/(x^{b}), 1/(b^{x}), 1/(e^{bx}), 1/(be^{x}), etc., where x is the length of sequence read corresponding to the nucleic acid remaining to be sequenced and b is a corrective parameter adjusting the ratio of the first subset of nucleotides or nucleotide analogs to the second subset of nucleotides or nucleotide analogs.

The functional relationship between the ratio of the first subset of nucleotides or nucleotide analogs to the second subset of nucleotides or nucleotide analogs and the reads that have been sequenced may also take the form 1/(y+f), where y is the length of the sequence read corresponding to the nucleic acid that has been sequenced and f is a corrective factor. When f is positive it acts as a dilution factor. When f is negative it acts as a concentrating factor. When f is a concentrating factor the magnitude off may be less than the value of y. The corrective factor, f, may take on integer and non-integer values. The corrective factor, f, may be used to fix a signal-to-noise ratio, to maintain the signal-to-noise ratio above a minimum threshold, and/or to target individual nucleotides, nucleotide analogs, read lengths, and/or read positions. Other functions relating the first subset of nucleotides or nucleotide analogs to the second subset of nucleotides or nucleotide analogs may be contemplated including 1/(gy), 1/(y^{g}), 1/(g^{y}), 1/(e^{gy}), 1/(ge^{y}), etc., where y is the length of sequence read corresponding to the nucleic acid that has been sequenced and g is a corrective parameter adjusting the ratio of the first subset of nucleotides or nucleotide analogs to the second subset of nucleotides or nucleotide analogs.

Functions relating the first subset of nucleotides or nucleotide analogs to the second subset of nucleotides or nucleotide analogs as a function of the length of sequence read corresponding to the nucleic acid remaining to be sequenced as described herein and functions relating the first subset of nucleotides or nucleotide analogs to the second subset of nucleotides or nucleotide analogs as a function of the length of sequence read corresponding to the nucleic acid that has been sequenced ad described herein may be combined in any manner, along with their respective corrective factors.

The ratio of the first subset of nucleotides or nucleotide analogs to the second subset of nucleotide analogs may not be the same the ratio incorporated or anticipated to be incorporated. As modified nucleotides or nucleotide analogs (such as those with detectable moieties, those with terminating subunits, or those with detectable moieties and terminating subunits).

The target nucleic acid molecule may be a deoxyribonucleic acid molecule. To generate the plurality of nucleic acid molecules the target nucleic acid molecule may be subjected to nucleic acid amplification. Such nucleic acid amplification may be polymerase chain reaction, emulsion-based amplification, bridge amplification, or any amplification technique known in the art.

Alternatively or in addition to, the target nucleic acid molecule may be a ribonucleic acid molecule. The target nucleic acid molecule may be subjected to reverse transcription to generate the plurality nucleic acid molecules.

The support may be a solid support, a biological support, a non-biological support, an organic support, an inorganic support, or any combination thereof. The support may be in optical communication with the detector, may be physically in contact with the detector, may be separated from the detector by a distance, or any combination thereof. The support may have a plurality of independently addressable locations. The nucleic acid molecules may be immobilized to the support at a given independently addressable location of the plurality of independently addressable locations. Immobilization of each of the plurality of nucleic acid molecules to the support may be aided by the use of an adaptor.

The first subset of nucleotides or nucleotide analogs may comprise nucleotides or nucleotide analogs that are each labeled with a detectable moiety. The detectable moiety, the nucleotide or nucleotide analogs, or any combination thereof may acoustically detectable, chemically detectable, electrically detectable (including detecting current, potential, or magnetism, including amplitudes and frequencies thereof), fluidically detective, mechanically detectable (such as through changes in force, pressure, proximity, etc.), optically detectable, radiologically detectable, or thermally detectable, or any combination of aforementioned detectabilities. In those instances wherein the detectable moiety is optically detectable the detectable moiety may be a fluorophore. Furthermore the detectable moiety may be an acceptor, a donor, both, or neither. The detectable moiety, nucleotide or nucleotide analog, or any combination thereof may detected via Förster resonance energy transfer (FRET).

The first subset of nucleotides or nucleotide analogs may each be terminated with a terminating subunit. The terminating subunit may be a detectable moiety of any type described herein. The terminating subunit may prevent or cause a reduction in rate of reaction of a next nucleotide or nucleotide analog to be incorporated or anticipated to be incorporated, as described elsewhere herein.

The method may further comprise cleaving, bleaching, quenching or disabling one or more detectable moieties. Cleaving, bleaching, quenching, or disabling of the detectable moiety may be subsequent to detecting the detectable moiety from the given nucleotide or nucleotide analog.

The first subset of nucleotides or nucleotide analogs may comprise nucleotides or nucleotide analogs that are each terminated with a terminating subunit. These nucleotides or nucleotide analogs of the first subset may each be labeled with a detectable moiety according to any type described herein. In some cases, the detectable moiety may be at least a portion of the terminating subunit.

The operations of directing the plurality of nucleotides or nucleotide analogs (themselves comprising at least a first subset of nucleotides or nucleotide analogs that are labeled and terminated and a second subset of nucleotides or nucleotide analogs that are unlabeled and unterminated) to the support, subjecting the plurality of nucleic acid molecules to an incorporation reaction under conditions that are sufficient to incorporate the first subset of nucleotides or nucleotide analogs and the second subset of nucleotides or nucleotide analogs into the plurality of nucleic acid molecules, and using the detector to detect the given nucleotide or nucleotide analog to determine the nucleic acid sequence of the target nucleic acid molecule may be repeated. This sequence may be repeated any number of times from zero to the number needed to determine the nucleic acid sequence. This sequence may be repeated without cleaving a terminating subunit of the given nucleotide or nucleotide analog. The plurality of nucleotides or nucleotide analogs may include bases of a first type and each repetition of the aforementioned operations of directing the plurality of nucleotides or nucleotide analogs to the support, subjecting the plurality of nucleic acid molecule to an incorporation reaction, and using the detector to detect the given nucleotide or nucleotide analogs may comprise an additional plurality of nucleotides or nucleotide analogs including bases of a second type different than the first type.

The given nucleotide or nucleotide analog may be detected while incorporating the given nucleotide or nucleotide analog into the given nucleic acid molecule. Alternatively or in combination, the given nucleotide or nucleotide analog may be detected subsequent to incorporating the given nucleotide or nucleotide analog into the given nucleic acid molecule.

In another aspect of the present disclosure, a method for sequencing a target nucleic acid molecule comprises subjecting a plurality of nucleic acid molecules exhibiting sequence homology to the target nucleic acid molecule to at most about 10,000, 5,000, 4,000, 3,000, 2,000, 1,000, 500, 400, 300, 200, 100, 50, 40, 30, 20, or 10 cycles of a nucleic acid extension reaction while measuring detectable signals from the plurality of nucleic acid molecules. Alternatively, the method may comprise subjecting the plurality of nucleic acid molecules exhibiting sequence homology to the target nucleic acid molecule to more than about 10,000 cycles of nucleic acid extension reaction while measuring detectable signals from the plurality of nucleic acid molecules. The detectable signals may correspond to individual nucleotides or nucleotide analogs incorporated into the plurality of nucleic acid molecules during the nucleic acid extension reaction. The detectable signals may be used to generate a sequence of the target nucleic acid molecule.

The sequence may have a length of at least about 5 bases, 10 bases, 20 bases, 30 bases, 40 bases, 50 bases, 100 bases, 200 bases, 300 bases, 400 bases, 500 bases, 600 bases, 700 bases, 800 bases, 900 bases, 1,000 bases, 1,100 bases, 1,200 bases, 1,300 bases, 1,400 bases, 1,500 bases, 1,600 bases, 1,700 bases, 1,800 bases, 1,900 bases, 2,000 bases, 3,000 bases, 4,000 bases, 5,000 bases, 10,000 bases, or more. The sequence may be generated at an accuracy of at least about at least about 50%, 60%, 70%, 80%, 90%, 95%, 96%, 97%, 98%, or 99% with or without resequencing. The sequence may be generated in the absence of read alignment.

### Systems for Nucleic Acid Sequencing

In another aspect of the present disclosure, a system for determining a nucleic acid sequence of a target nucleic acid molecule may comprise a support for immobilizing a plurality nucleic acid molecules operatively coupled to a detector and a controller with one or more computer processors individually or collectively programmed to direct a plurality of nucleotides or nucleotide analogs to the support and use the detector to detect a detectable moiety from the given nucleotide or nucleotide analogs, thereby determining the nucleic acid sequence of the target nucleic acid molecule.

Each of the plurality of nucleic acid molecules may exhibit sequence homology to the target nucleic acid molecule. Each of the plurality of nucleic acid molecules may exhibit sequence identity to the target nucleic acid molecule. Each of the plurality of nucleic acid molecules may exhibit sequence complementarity to the target nucleic acid molecule.

The plurality of nucleotides or nucleotide analogs may comprise at least a first subset of nucleotides or nucleotide analogs with a detectable moiety and a terminating subunit and a second subset of nucleotides or nucleotide analogs in which none of the nucleotides or nucleotide analogs comprises a detectable moiety and the terminating subunit. The plurality of nucleotides or nucleotide analogs may be incorporated into the plurality nucleic acid molecules. During incorporation, a given nucleotide or nucleotide analog from either the first subset of nucleotides or nucleotide analogs or the second subset of nucleotides or nucleotide analogs may be incorporated into a given nucleic acid molecule from the plurality of nucleic acid molecules. At least a portion of the first subset of nucleotides or nucleotide analogs may be incorporated into the given nucleic acid molecule and thereby giving the given nucleotide or nucleotide analog a detectable moiety and terminating subunit. The detector may be used to detect the detectable moiety from the given nucleotide or nucleotide analog and thus determine the nucleic acid sequence of the target nucleic acid molecule. The system may further comprise the detector.

Such a system may comprise a support that is a chip or is part of a chip. Alternatively or in addition to, the controller may be part of the chip. The support may be optical communication with the detector. The support may physically contact the detector. The support and the detector may be separated by a distance. The distance between the support may be constant or variable, wherein the variability may be a function of the intensity of the response, the field of view required, the time eclipsed during sequencing, the total read length, the remaining sequence to be read, or the anticipated length of the sequence to be read, or any combination thereof. The support of any embodiment may have a plurality of independently addressable locations.

In some examples, the support is integrated with or adjacent to a waveguide for delivering excitation energy (e.g., optical excitation energy). As an alternative, the waveguide may be configured to capture an emitted signal during sequencing, such as fluorescence. The waveguide may be adjacent to or integrated with a chip.

In another aspect of the present disclosure, a system for determining a nucleic acid sequence of a target nucleic acid molecule comprises a support for immobilizing a plurality nucleic acid molecules (each of the plurality of nucleic acid molecules may exhibit sequence homology to the target nucleic acid molecule) operatively coupled to a detector and a controller, the controller comprising one or more computer processors that may be individually or collectively programmed to direct a plurality of nucleotides or nucleotide analogs to the support, the plurality of nucleotides or nucleotide analogs comprising at least a first subset of nucleotides or nucleotide analogs that are labeled and terminated and a second subset of nucleotides or nucleotide analogs that are unlabeled and unterminated, subjecting the plurality of nucleic acid molecules to an incorporation reaction under conditions that are sufficient to incorporate the first subset of nucleotides or nucleotide analogs and the second subset of nucleotides or nucleotide analogs into the plurality of nucleic acid molecules, and using the detector to detect the given nucleotide or nucleotide analog, thereby determining the nucleic acid sequence of the target nucleic acid molecule. During incorporation, the given nucleotide or nucleotide analog from the first subset of nucleotides or nucleotides analogs may be incorporated into the given nucleic acid molecule from the plurality of nucleic acid molecules, thus rendering the given nucleotide or nucleotide analog labeled and terminated. The system may further comprise the detector.

Such a system may comprise a support that is part of a chip. Alternatively or in addition to, the controller may be part of the chip. The support may be in optical communication with the detector. The support may physically contact the detector. The support may be in proximity of the detector. The support and the detector may be separated by a distance. The distance between the support may be constant or variable, wherein the variability may be a function of the intensity of the response, the field of view required, the time eclipsed during sequencing, the total read length, the remaining sequence to be read, or the anticipated length of the sequence to be read, or any combination thereof. The support of any embodiment may have a plurality of independently addressable locations.

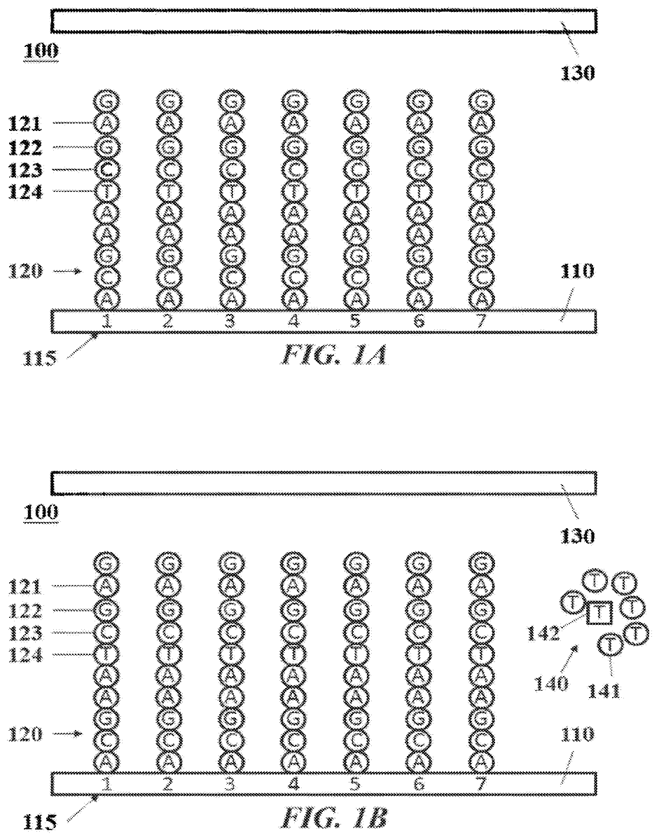

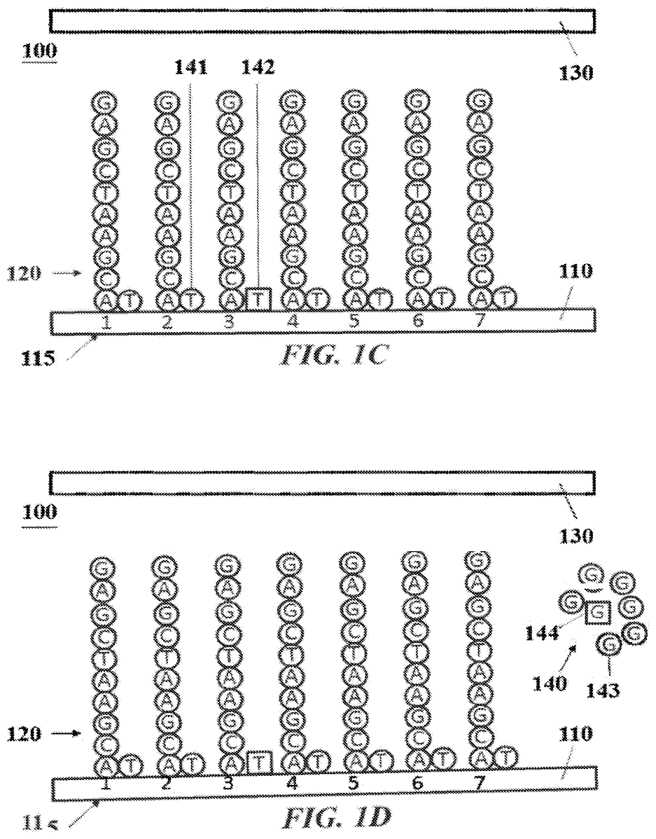

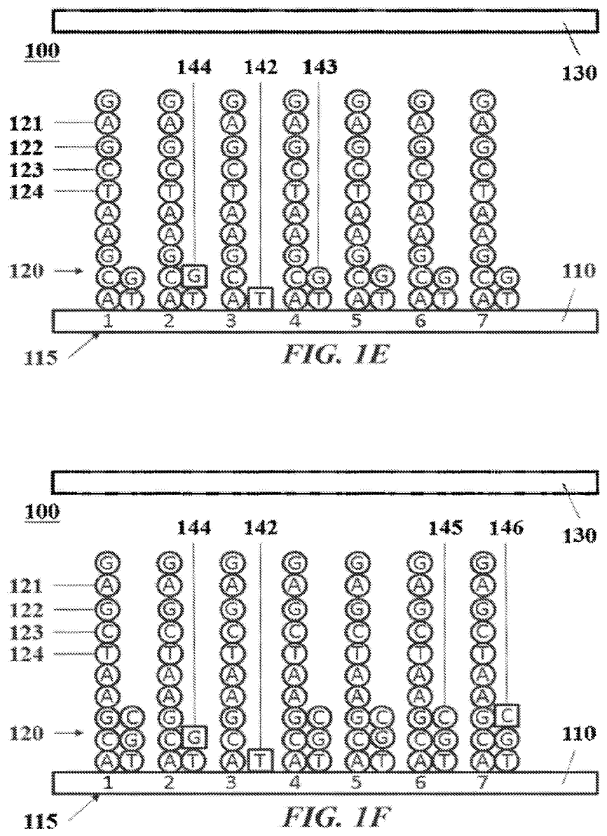

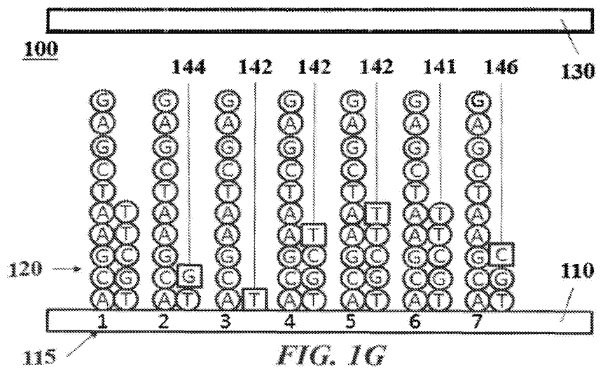

FIGS. 1A-1G schematically illustrate an example system 100 and method for sequencing a nucleic acid molecule. The system 100 may comprise a support 110, a detector 130, and a sub-system, module or unit (not illustrated) by which to introduce a plurality of nucleotides or nucleotide analogs. A plurality of nucleic acid molecules 120 may be immobilized on the support 110 at support locations 115 (e.g., locations 1, 2, 3, 4, 5, 6, 7). The support locations 115 may be predetermined or predefined. The support locations 115 may be selected or specific for each, a subset, or all of the nucleic acid molecules 120. The support locations 115 may be independently or individually addressable. The support 110 may be of any type described herein such as a slide, a bead, a resin, a chip, an array, a matrix, a membrane, a nanopore, a gel, a bead on a flat substrate (such as glass, plastic, silicon, metal, etc.), or a bead within a well of a substrate. The support 110 may in optical, electrical, mechanical, and/or thermal communication with the detector 130, may be optically, electrically, or mechanically coupled to the detector 130, may be physically in contact with the detector 130, may be in proximity of the detector 130, may be separated from the detector 130 by a distance, or any combination thereof. The support locations 115 may comprise a plurality of independently addressable locations and nucleic acid molecules 120 may be immobilized to the support 110 at a given independently addressable location of the plurality of independently addressable locations (such as, for example, those illustrated to be immobilized at the independently addressable locations labeled 1, 2, 3, 4, 5, 6, and 7 in FIGS. 1A-1G). Immobilization of each of the plurality of nucleic acid molecules 120 to the support 110 may be aided by the use of an adaptor (not illustrated).

FIGS. 1A-1G illustrate a system 100 with seven nucleic acid molecules 120 immobilized on the support 110 at seven support locations 115. Each of the plurality of nucleic acid molecules 120 may be immobilized on the support either directly or through a linker. The plurality of nucleic acid molecules 120 may have the same sequence or substantially the same sequence. As an alternative, the plurality of nucleic acid molecules 120 may have different sequences. The plurality of nucleic acid molecules 120 may be copies of a template or multiple templates. Although seven nucleic acid molecules are illustrated, any number of nucleic acid molecule(s) may be used. For example, the system 100 may have at least about 1, 2, 3, 4, 5, 6, 7, 8, 9, 10, 20, 30, 40, 50, 100, 200, 300, 400, 500, 1,000, 10,000, 50,000, 100,000, 500,000, 1,000,000, or more nucleic acid molecules 120 immobilized on the support 110, or any value in between any two of the values listed. The plurality of nucleic acid molecules 120 may be copies of a template nucleic acid molecule or multiple template nucleic acid molecules. The system 100 may comprise at most about 1,000,000, 500,000, 100,000, 50,000, 10,000, 1,000, 500, 400, 300, 200, 100, 50, 40, 30, 20, 10, 9, 8, 7, 6, 5, 4, 3, 2, or 1 nucleic acid molecule(s) 120 immobilized on the support 110, or the number of copies may take on any value in between any two of the values listed.

The support 110 may comprise at least about 1, 5, 10, 50, 100, 500, 1,000, 5,000, 10,000, 50,000, 100,000, 500,000, 1,000,000, 5,000,000, 10,000,000, 50,000,000, 100,000,000, 500,000,000, 1,000,000,000, or more support locations 115, or any value in between any two of the values listed. The support 110 may comprise at most about 1,000,000,000, 500,000,000, 100,000,000, 50,000,000, 10,000,000, 5,000,000, 1,000,000, 500,000, 100,000, 50,000, 10,000, 5,000, 1,000, 500, 100, 50, 10, 5, 1 support location(s) 115, or any value in between any two of the values listed. The support locations 115 may each be individually addressable. The support locations 115 may comprise a subset of support locations that are individually addressable. The support locations 115 may comprise a first subset of support locations that are individually addressable and a second subset of support locations that are not individually addressable. The support locations 115 may comprise one or more subsets of support locations that are individually addressable. The support locations 115 may comprise one or more subsets of support locations that are individually addressable and each of the one or more subsets of support locations may be distinctively addressable from each of the other subsets of support locations from the one or more subsets of support locations.

The plurality of nucleic acid molecules 120 may comprise any number of nucleotides or nucleotide analogs. For example, a given nucleic acid molecule of the plurality of nucleic acid molecules can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, or 50 kb, or more. The nucleotides or nucleotide analogs may be of the same type (e.g., all adenine) or different types (e.g., adenine and guanine). Although four different types of nucleotides or nucleotide analogs have been illustrated (adenine 121, guanine 122, cytosine 123, and thymine 124), any number of types nucleotides or nucleotides analogs may be used, such as, for example, at least one type of nucleotide or nucleotide analog, two types of nucleotides or nucleotide analogs, three types of nucleotides or nucleotide analogs, four types of nucleotides or nucleotide analogs, five types of nucleotides or nucleotide analogs, six types of nucleotides or nucleotide analogs, seven types of nucleotides or nucleotide analogs, eight types of nucleotides or nucleotide analogs, nine types of nucleotides or nucleotide analogs, ten types of nucleotides or nucleotide analogs, or more.

FIG. 1B shows a mixture 140 of a first set of nucleotides or nucleotide analogs comprising a first subset of nucleotides or nucleotide analogs 142 of a first type (illustrated with a square outline) and a second subset of nucleotides or nucleotide analogs 141 of a second type (illustrated with a circular outline). The nucleotide or nucleotide analogs 142 of the first type may differ from the nucleotide or nucleotide analogs 141 of the second type. For example, the nucleotide or nucleotide analogs 142 of the first type may comprise a detectable moiety, a terminating subunit, or both and the nucleotide or nucleotide analogs 141 of the second type may not comprise a detectable moiety, a terminating subunit, or either.

The mixture 140 may be introduced to the system 100 through various approaches, such as a fluid flow system (e.g., microfluidic fluid flow system), such as with aid of a controller (not shown). The ratio of the first type of nucleotide or nucleotide analogs 142 to the second type of nucleotide or nucleotide analogs 141 may be in accordance with any manner described herein.

FIG. 1C shows the nucleotide or nucleotide analogs of the mixture 140 incorporated at the locations 115 of the plurality of nucleic acid molecules 120. Some of the plurality of nucleic acid molecules 120 immobilized on the support 110 may incorporate nucleotide or nucleotide analogs 142 of the first type and some of the plurality of nucleic acid molecules 120 immobilized to the support 110 may incorporate nucleotide or nucleotide analogs 141 of the second type. Some of the plurality of nucleic acid molecules 120 immobilized to the support 110 may not incorporate nucleotide or nucleotide analogs of either the first type or the second type. The detector 130 may detect nucleotide or nucleotide analogs 142 of the first type through a detectable moiety.

FIG. 1D shows the introduction of the mixture 140 comprising a second of set of nucleotide or nucleotide analogs 144 of a first type and a second set of nucleotide or nucleotide analogs 143 of a second type. The second set of nucleotide or nucleotide analogs 144 of the first type may be chemically distinct from the first set of nucleotide or nucleotide analogs 142 of the first type seen in the previous illustration. The second set of nucleotide or nucleotide analogs 144 of the first type may comprise a different nucleobase than the first set of nucleotide or nucleotide analogs 142 of the first type seen in the previous illustration. The second set of nucleotide or nucleotide analogs 144 of the first type may differ from the second set of nucleotide or nucleotide analogs 143 of the second type. For example, the nucleotide or nucleotide analogs 144 of the first type may comprise a detectable moiety, a terminating subunit, or both and the nucleotide or nucleotide analogs 143 of the second type may not comprise a detectable moiety, a terminating subunit, or either.

FIG. 1E shows the incorporation of the nucleotide or nucleotide analogs of the mixture 140 at the locations 115 of the plurality of nucleic acid molecules 120. The first set of nucleotide or nucleotide analogs of the first type 142 (as seen at location 3) may comprise a terminating subunit and thereby not allow further incorporation of nucleotide or nucleotide analogs from the mixture 140 into the plurality of nucleic acid molecules 120 immobilized on the support 110. Alternatively or in addition, such as when the nucleotide or nucleotide analog of the first type 142 (as seen at location 3) is a virtual terminator and has an unblocked 3′ hydroxyl group, the terminating subunit may not completely prevent but reduce a rate of incorporation of a next nucleotide or nucleotide analog anticipated to be incorporated. Nucleotide or nucleotide analogs 144 of the first type from the second set of nucleotide or nucleotide analogs and nucleotide or nucleotide analogs 143 of the second type from the second set of nucleotide or nucleotide analogs from the mixture 140 may be incorporated at any of the plurality of nucleic acid molecules 120 immobilized at the individually addressable locations 115 of the support 110 not previously occupied or (perfectly) terminated. In this illustrated non-limiting example, the second set of nucleotide or nucleotide analogs 144 of the first type are incorporated at location 2 of the support 110 while second set of nucleotide or nucleotide analogs 143 of the second type are incorporated at all locations not occupied by nucleotide or nucleotide analogs with a (perfectly) terminating subunit (at locations 1, 4, 5, 6, 7 of this illustration).

FIG. 1F shows the incorporation of a third set of nucleotide or nucleotide analogs into the plurality of nucleic acid molecules 120 immobilized on the support 110. This third set of nucleotide or nucleotide analogs may comprise nucleotide or nucleotide analogs 146 of a first type and nucleotide or nucleotide analogs 145 of a second type. The third set of nucleotide or nucleotide analogs 146 of the first type may differ from the third set of nucleotide or nucleotide analogs 145 of the second type. For example, the nucleotide or nucleotide analogs 146 of the first type may comprise a detectable moiety, a terminating subunit, or both and the nucleotide or nucleotide analogs 141 of the second type may not comprise a detectable moiety, a terminating subunit, or either.

The detector 130 may detect the detectable moieties, terminating subunits, or both of the nucleotide or nucleotide analogs of the first set of nucleotide or nucleotide analogs 142 of the first type, the second set of nucleotide or nucleotide analogs 144 of the first type, or the third set of nucleotide or nucleotide analogs 146 of the first type, or any combination thereof, individually or collectively at any time during any of the methods described herein. The detector 130 may detect the detectable moieties, terminating subunits, or both of the nucleotides or nucleotide analogs during or subsequent to incorporation. In an example, the detector 130 may detect the first set of nucleotide or nucleotide analogs 142 of the first type, introduce a new mixture 140 then detect the second set of nucleotide or nucleotide analogs 144 of the first type, introduce a new mixture 140 then detect the third set of nucleotide or nucleotide analogs 146 of the first type, etc. The detector 130 may also detect any possibly detectable moiety, terminating subunit, or both of all available sets of nucleotide or nucleotide analogs of the first type. For example, an initial mixture 140 may be introduced to the system 100 and the detector 130 attempts to detect all available detectable sets of nucleotide or nucleotide analogs such that even if only the first set of nucleotide or nucleotide analogs has been introduced, the detector 130 may attempt to detect the first set of nucleotide or nucleotide analogs 142 of the first type, the second set of nucleotide or nucleotide analogs 144 of the first type, the third set of nucleotide or nucleotide analogs 146 of the first type, etc. simultaneously, sequentially, or concurrently before the introduction of the next set of nucleotide or nucleotide analogs.

Any number of sets of nucleotides or nucleotide analogs may be used, including, but not limited to, at least about 1, 2, 3, 4, 5, 6, 7, 8, 9, 10, 20, 30, 40, 50, 100, 500, 1,000, 5,000, 10,000, 50,000, 100,000, 500,000, 1,000,000, 5,000,000, 10,000,000, 50,000,000, 100,000,000, 500,000,000, 1,000,000,000 or more sets of nucleotides or nucleotide analogs, or any value in between those listed. The number of sets of nucleotides and nucleotide analogs may be at most about 1,000,000,000, 500,000,000, 100,000,000, 50,000,000, 10,000,000, 5,000,000, 1,000,000, 500,000, 100,000, 50,000, 10,000, 5,000, 1,000, 500, 100, 50, 40, 30, 20, 10, 9, 8, 7, 6, 5, 4, 3, 2, or 1 set(s) of nucleotides or nucleotide analogs, or any value in between those listed. Sets of nucleotide or nucleotide analogs may differ from each other by each set comprising distinct nucleobases, by each set comprising distinct dyes, by each set comprising dyes of distinct colors, by each comprising distinct combinations and/or ratios of first and second subsets of nucleotide or nucleotide analogs as described herein (such as by having distinct dilution or concentration ratios), or any combination thereof.

FIG. 1G shows an example of the incorporation of a fourth set of nucleotide or nucleotide analogs that is the same as the first set of nucleotide or nucleotide analogs. This illustrates an effectively linear homopolymer signal.

Though FIGS. 1A-1G schematically illustrate an example of the incorporation and detection of single nucleotide or nucleotide analogs of a type detectable by the detector 130 (consider the nucleotide or nucleotide analogs of 142, 144, and 146, for example), one or more nucleotides or nucleotide analogs of a type detectable by the detector 130 may be detected at any given time. Given the statistical nature of incorporation, the number of nucleotides or nucleotide analogs of a type detectable by the detector 130 may vary from operation to operation, may vary with concentration, or may vary stochastically. As a non-limiting, illustrative example, with a system having 100 copies of a target nucleic acid sequence subjected to a mixture comprising 1% of nucleotides or nucleotide analogs with a detectable moiety (for instance, labeled deoxynucleotides), one may expect on average for 1 copy of the target nucleic acid sequence to incorporate a nucleotide or nucleotide analog with a detectable moiety. However, the statistical nature of the incorporation may mean that 0, or 2, or 3, etc. nucleotide or nucleotide analogs with a detectable moiety may be incorporated into the copies of the target nucleic acid sequence. As another non-limiting, illustrative example, the system may have 100,000 copies of a target nucleic acid sequenced subjected to a mixture comprising 1% of nucleotides or nucleotide analogs with a detectable moiety. In this latter example, 1,000 copies of the target nucleic acid may be expected, on average, to incorporate nucleotides or nucleotide analogs with detectable moieties. In both cases, there may be a variance. That variance may be proportional to or a function of the square root of the total number of copies in the system. The variance may be proportional to or a function of the square root of the total number of copies remaining to be incorporated in the system. The variance may be proportional to or a function of the square root of the total number of copies that have been incorporated in the system in subsequent iterations. Statistical methods may be employed by a computer control system to determine the number of incorporations of the nucleotides or nucleotide analogs with a detectable moiety.

Though FIGS. 1A-1G illustrate nucleotides or nucleotide analogs comprising a terminating subunit that are true terminators and completely prevent further incorporation of the next nucleotide or nucleotide analog in the sequence, the systems and methods described herein may apply to nucleotides or nucleotide analogs comprising a terminating subunit that are imperfect terminators (e.g., a virtual terminator that has an unblocked 3′ hydroxyl group) that reduce the rate of incorporation of the next nucleotide or nucleotide analog in the sequence. In some cases, the detector 130 may detect at different time points or in real-time the signals indicative of incorporation of consecutive nucleotides or nucleotide analogs for a sequence. Alternatively or in addition, a controller (not shown) in communication with the detector 130 may use detection or measurement data from the detector 130 to determine the respective rates of incorporation (or substantial incorporation such that a signal is detected). The signals indicative of incorporation, if any, can be compared to predetermined signals for a particular type of nucleotide or nucleotide analog (terminator) to determine the sequence. In some instances, a difference or ratio between consecutive signals can be compared. The comparison can be linear or non-linear. In some instances, a difference or ratio between non-consecutive signals can be compared. In some instances, a different type of computation (e.g., mean) can be performed by the controller.

### Computer Control Systems

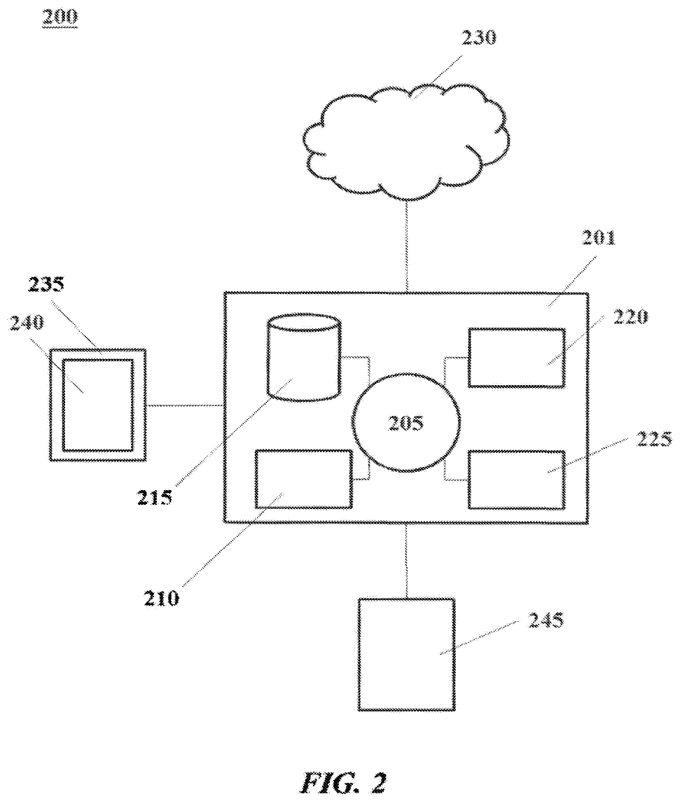

The present disclosure provides computer control systems that are programmed to implement methods of the disclosure. FIG. 2 shows a system 200 comprising a computer system 201 that is programmed or otherwise configured to implement nucleic acid sequencing methods and systems of the present disclosure. The computer system 201 can regulate various aspects of sequencing of the present disclosure, such as, for example, directing sequencing of a nucleic acid molecule and/or determining a sequence of the nucleic acid molecule. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user (e.g., health care provider). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, sequence information to a user, or for enabling a user to sequence a nucleic acid molecule. Examples of UI's include, without limitation, a graphical user interface (GUI) and web-based user interface.

The system 200 can also include a nucleic acid sequencing system 245, which can sequence a nucleic acid molecule in the manner described elsewhere herein. The nucleic acid sequencing system 245 can include (i) one or more units for sample preparation and (ii) a sequence unit to generate a nucleic sequence or multiple sequences (e.g., reads) of the nucleic acid molecule.

Methods and systems of the present disclosure can be implemented by way of one or more algorithms. An algorithm can be implemented by way of software upon execution by the central processing unit 205. The algorithm can, for example, perform sequence alignment to generate a consensus sequence.

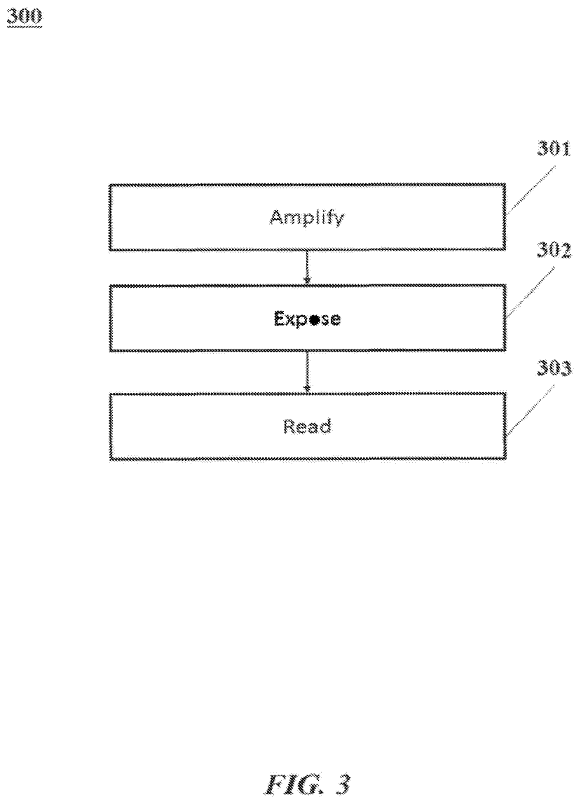

FIG. 3 shows a method for sequencing a nucleic acid molecule. The method 300 shows the sequential operations of amplifying 301, incorporation 302, and reading 303 the target nucleic acid. Amplification 301 may be of any sort described herein. Amplifying 301 may consist of a low cost, high copy method, such as emulsion PCR (ePCR), wherein a target molecule is denatured, annealed (a reverse strand anneals to the adapter site on a bead, for instance, while a primer anneals to a forward strand), and extended (polymerase amplifies the forward strand starting from the bead towards the primer site while the reverse strand starts from the primer towards the bead). This cycle of denaturing, annealing, and extending may be repeated any number of times. The cycle of denaturing, annealing, and extending to amplify 301 a target may be repeated at least about 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 200, 300, 400, 500, 1,000 times before proceeding to the next operation. Incorporation 302 of the target molecule may be via any method described herein (utilizing ddNTPs, enzymes, etc.), such as introducing a mixture comprising a first subset of nucleotides or nucleotide analogs and a second subset of nucleotides or nucleotide analogs. Reading 303 the sequence of the target molecule may be of any sort described herein. Such a method may result in a lower cost per base read (for instance by using standard polymerase without replenishment), a shorter read cycle (as the sequencing procedure of labelling, washing, and reading can result in much faster incorporations with 99% natural nucleotides), less systematic errors (by, for example, leverage natural DNA strands with single reporters created using the methods described herein) and allow longer sequences to be read (for instance, by using stable, long nucleic acid sequences constructed with all natural nucleotides and a single terminating label).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-95. (canceled)

96. A method for determining a nucleic acid sequence of a target nucleic acid molecule, comprising:
(a) immobilizing a plurality of nucleic acid molecules to a support, wherein each of said plurality of nucleic acid molecules exhibits sequence homology to said target nucleic acid molecule, and wherein said support is operatively coupled to a detector;
(b) directing a plurality of nucleotides or nucleotide analogs to said support, wherein said plurality of nucleotides or nucleotide analogs comprises at least a first subset of nucleotides or nucleotide analogs and a second subset of nucleotides or nucleotide analogs, wherein (i) said first subset of nucleotides or nucleotide analogs comprises nucleotides or nucleotide analogs that are labeled and terminated, and (ii) said second subset of nucleotides or nucleotide analogs comprises nucleotides or nucleotide analogs that are unlabeled and unterminated;
(c) subjecting said plurality of nucleic acid molecules to an incorporation reaction under conditions that are sufficient to incorporate said first subset of nucleotides or nucleotide analogs and said second subset of nucleotides or nucleotide analogs into said plurality of nucleic acid molecules, wherein during incorporation, a given nucleotide or nucleotide analog from said first subset of nucleotides or nucleotide analogs is incorporated into a given nucleic acid molecule from said plurality of nucleic acid molecules, which given nucleotide or nucleotide analog is labeled and terminated; and
(d) using said detector to detect said given nucleotide or nucleotide analog, thereby determining said nucleic acid sequence of said target nucleic acid molecule.

97. The method of claim 96, wherein a ratio of said first subset of nucleotides or nucleotide analogs to said second subset of nucleotides or nucleotide analogs is less than 50%.

98. The method of claim 96, wherein said target nucleic acid molecule is a deoxyribonucleic acid molecule.

99. The method of claim 96, further comprising subjecting said target nucleic acid molecule to nucleic acid amplification to generate said plurality nucleic acid molecules.

100. The method of claim 96, wherein said target nucleic acid molecule is a ribonucleic acid molecule.

101. The method of claim 96, wherein said support is a solid support, biological support, non-biological support, organic support, inorganic support, or any combination thereof.

102. The method of claim 96, wherein said first subset of nucleotides or nucleotide analogs comprises nucleotides or nucleotide analogs that are each labeled with a detectable moiety.

103. The method of claim 96, further comprising cleaving, bleaching, quenching or disabling said detectable moiety.

104. The method of claim 96, wherein said first subset of nucleotides or nucleotide analogs comprises nucleotides or nucleotide analogs that are each terminated with a terminating subunit.

105. The method of claim 96, wherein in (d), said given nucleotide or nucleotide analog is detected via Förster resonance energy transfer (FRET).

106. The method of claim 96, further comprising repeating (b)-(d).

107. The method of claim 96, wherein said plurality of nucleotides or nucleotide analogs is incorporated using a nucleic acid polymerizing enzyme.

108. The method of claim 96, wherein said given nucleotide or nucleotide analog is detected while incorporating said given nucleotide or nucleotide analog into said given nucleic acid molecule.

109. The method of claim 96, wherein said given nucleotide or nucleotide analog is detected subsequent to incorporating said given nucleotide or nucleotide analog into said given nucleic acid molecule.

110. The method of claim 96, wherein said support is in optical communication with said detector.

111. The method of claim 96, wherein said support has a plurality of independently addressable locations.

112. The method of claim 96, wherein each of said plurality nucleic acid molecules is immobilized to said support using an adaptor.

113. A method for sequencing a target nucleic acid molecule, comprising (a) subjecting a plurality of nucleic acid molecules exhibiting sequence homology to said target nucleic acid molecule to at most 4000 cycles a nucleic acid extension reaction while measuring detectable signals from said plurality of nucleic acid molecules, which detectable signals correspond to individual nucleotides or nucleotide analogs incorporated into said plurality of nucleic acid molecules during said nucleic acid extension reaction, and (b) using said detectable signals to generate a sequence of said target nucleic acid molecule at a length of at least about 500 bases and an accuracy of at least about 97%.

114. A system for determining a nucleic acid sequence of a target nucleic acid molecule, comprising:
a support for immobilizing a plurality nucleic acid molecules, wherein each of said plurality of nucleic acid molecules exhibits sequence homology to said target nucleic acid molecule, and wherein said support is operatively coupled to a detector; and
a controller comprising one or more computer processors that are individually or collectively programmed to:
(a) direct a plurality of nucleotides or nucleotide analogs to said support, which plurality of nucleotides or nucleotide analogs comprises at least a first subset of nucleotides or nucleotide analogs and a second subset of nucleotides or nucleotide analogs, wherein (i) each of said first subset of nucleotides or nucleotide analogs comprises a detectable moiety and a terminating subunit, and (ii) none of said second subset of nucleotides or nucleotide analogs comprises said detectable moiety and said terminating subunit, wherein said plurality of nucleotides or nucleotide analogs are incorporated into said plurality nucleic acid molecules, wherein during incorporation, a given nucleotide or nucleotide analog from said first subset of nucleotides or nucleotides analogs is incorporated into a given nucleic acid molecule from said plurality of nucleic acid molecules, which given nucleotide or nucleotide analog comprises said detectable moiety and said terminating subunit; and
(b) use said detector to detect said detectable moiety from said given nucleotide or nucleotide analog, thereby determining said nucleic acid sequence of said target nucleic acid molecule.

115. A system for determining a nucleic acid sequence of a target nucleic acid molecule, comprising:
a support for immobilizing a plurality nucleic acid molecules, wherein each of said plurality of nucleic acid molecules exhibits sequence homology to said target nucleic acid molecule, and wherein said support is operatively coupled to a detector; and
a controller comprising one or more computer processors that are individually or collectively programmed to:
(a) direct a plurality of nucleotides or nucleotide analogs to said support, wherein said plurality of nucleotides or nucleotide analogs comprises at least a first subset of nucleotides or nucleotide analogs and a second subset of nucleotides or nucleotide analogs, wherein (i) said first subset of nucleotides or nucleotide analogs comprises nucleotides or nucleotide analogs that are labeled and terminated, and (ii) said second subset of nucleotides or nucleotide analogs comprises nucleotides or nucleotide analogs that are unlabeled and unterminated;
(b) subject said plurality of nucleic acid molecules to an incorporation reaction under conditions that are sufficient to incorporate said first subset of nucleotides or nucleotide analogs and said second subset of nucleotides or nucleotide analogs into said plurality of nucleic acid molecules, wherein during incorporation, a given nucleotide or nucleotide analog from said first subset of nucleotides or nucleotides analogs is incorporated into a given nucleic acid molecule from said plurality of nucleic acid molecules, which given nucleotide or nucleotide analog is labeled and terminated; and
(c) use said detector to detect said given nucleotide or nucleotide analog, thereby determining said nucleic acid sequence of said target nucleic acid molecule.


SEQUENCE LISTING (1 sequence)

SEQ ID NO: 1 - DNA, 10 bp, synthetic construct
  misc_feature 1..10: note = Description of Artificial Sequence: Synthetic oligonucleotide
  source 1..10: mol_type = other DNA
gagctaagca